(19) United States
(12) Patent Application Publication
(10) Pub. No.: US 2018/0127464 A1
(43) Pub. Date: May 10, 2018

(54) NOVEL EPHA4 INHIBITORS TARGETING ITS LIGAND BINDING DOMAIN

(71) Applicant: Sanford-Burnham Medical Research Institute, La Jolla, CA (US)

(72) Inventors: Maurizio PELLECCHIA, La Jolla, CA (US); Bainan WU, La Jolla, CA (US); Surya DE, La Jolla, CA (US)

(21) Appl. No.: 15/569,721

(22) PCT Filed: Apr. 29, 2016
(86) PCT No.: PCT/US16/30070

§ 371 Date: Oct. 26, 2017

Related U.S. Application Data
(60) Provisional application No. 62/154,670, filed on Apr. 29, 2015.

(51) Int. Cl. C07K 5/097; A61P 21/00; A61P 7/00; A61P 35/00; A61P 25/00; A61P 25/28

(52) U.S. Cl. CPC C07K 5/0821; A61P 21/00; A61P 7/00; A61K 38/00; A61P 25/00; A61P 25/28; A61P 35/00

(57) ABSTRACT

Provided herein are compounds and pharmaceutical compositions comprising EphA4 inhibitors. The EphA4 inhibitors and compositions thereof are useful for the treatment of ALS.

### CROSS REFERENCE

This application claims the benefit of U.S. Provisional Application No. 62/154,670 filed Apr. 29, 2015, the content of which is hereby incorporated by reference in its entirety.

### STATEMENT AS TO FEDERALLY SPONSORED RESEARCH

This invention was made with the support of the United States government under contract number CA138390 by the National Institute of Health (NIH). The government has certain rights in the invention.

### BACKGROUND

ALS is a progressive degenerative disease that affects motor neurons. Mutations in SOD1 (superoxide dismutase 1) and in chromosome 9 seem the most prevalent in those affected by the disease. Recently, a zebrafish model for ALS was developed by expression of SOD1 in embryos resulting in shorter and aberrantly branched motor. This model has been used to study and identify disease-modifying genes, revealing that the gene Rtk2 (the zebrafish equivalent of human EphA4) is critical for the onset and progression of the disease. Rtk2 knockdown in mutant-SOD1 zebrafish rescued the motor axonopathy induced by three different SOD1 mutations (A4V, G93A, G37R), without affecting axonal length. Despite tremendous efforts to identify contributing factors for ALS, the mechanisms underlying motor neuron death have not yet been fully elucidated and consequently no effective treatment is currently available for ALS, despite several clinical trials based on drugs from animal studies, which have been conducted and ultimately failed. The zebrafish studies clearly point at the EphA4 as a possible target. Pharmacological inhibition of the EphA4 via an intra-cerebroventricular administration in ALS rats overexpressing human mutant SOD1 (G93A) with an EphA4-blocking peptide, that was recently reported to bind to the EphA4-ligand binding domain (LBD) enhancing recovery and axonal sprouting in models of spinal cord injury, delayed ALS onset and prolonged survival. Moreover, in humans with ALS, EphA4 expression correlates inversely with disease onset and overall survival, while loss-of-function mutations in EphA4 are associated with long survival. These studies clearly suggest the EphA4 is a potential target for ALS and that targeting its ligand-binding domain provides a possible avenue to novel and effective therapeutics.

### BRIEF SUMMARY OF THE INVENTION

This disclosure provides, for example, compounds and compositions which are EphA4 inhibitors, and their use as medicinal agents, processes for their preparation, and pharmaceutical compositions that include disclosed compounds as at least one active ingredient. In some embodiments, the EphA4 inhibitors target the ligand binding domain of EPhA4.

One aspect provides a compound of formula (I), or a pharmaceutically acceptable salt, solvate, or stereoisomer thereof:

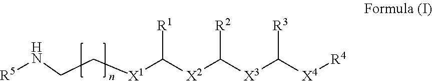

wherein
X¹, X², X³, and X⁴ are each independently —C(═O)NH—, —NHC(═O)—, —S(═O)₂NH—, —O—, —CH₂—, —CH₂CH₂—, —OCH₂—, —CH₂O—, —S—, —S(═O)₂—, or —C(═O)NHS(═O)₂—;
R¹, R², R³, and R⁴ are each independently alkyl, heterocycloalkyl, or cycloalkyl; wherein the alkyl, heterocycloalkyl, and cycloalkyl are optionally substituted with one of more R^{a};
R⁵ is —H, —CH₃, —C₂H₅, —C(═O)H, —C(═O)aryl, —C(═O)alkyl, or —C(═O)(CH₂CH₂O)ₖCH₃;
R⁶ is —H, halogen, alkyl, haloalkyl, alkoxy, haloalkoxy, —OH, —NH₂, S(═O)₂NH₂, —C(═O)NH₂, —NHC(═O)H, —NHC(═O)CH₃, —NO₂, —S(═O)CH₃, —NHS(═O)₂CH₃, —OCH₂OCH₃, —OCH₂CH₂OCH₃, —NHC(═O)H, or —NHC(═O)CH₃;
R^{a} is R⁶, aryl or heteroaryl; wherein the aryl or heteroaryl are optionally substituted with one or more R^{b};
R^{b} is —H, halogen, alkyl, haloalkyl, alkoxy, haloalkoxy, —OH, —NH₂, —S(═O)₂NH₂, —C(═O)NH₂, —NHC(═O)H, —NHC(═O)CH₃, —NO₂, —S(═O)CH₃, —NHS(═O)₂CH₃, —OCH₂OCH₃, or —OCH₂CH₂OCH₃;
n is 0-4; and
k is 1-1000.

In some embodiments of a compound of Formula (I), or a pharmaceutically acceptable salt, solvate, or stereoisomer thereof, n is 2-4.

In some embodiments of a compound of Formula (I), or a pharmaceutically acceptable salt, solvate, or stereoisomer thereof, R¹, R², R³, and R⁴ are each independently alkyl substituted with one of more R^{a}.

In some embodiments of a compound of Formula (I), or a pharmaceutically acceptable salt, solvate, or stereoisomer thereof, R¹, R², R³, and R⁴ are each independently —CH₃ or —CH₂CH₃ substituted with one of more R^{a}.

In some embodiments of a compound of Formula (I), or a pharmaceutically acceptable salt, solvate, or stereoisomer thereof, R¹, R², R³, and R⁴ are each independently —CH₃ or —CH₂CH₃ substituted with one R^{a}.

In some embodiments of a compound of Formula (I), or a pharmaceutically acceptable salt, solvate, or stereoisomer thereof, R¹, R², R³, and R⁴ are each independently —CH₃ or —CH₂CH₃ substituted with two R^{a}.

In some embodiments of a compound of Formula (I), or a pharmaceutically acceptable salt, solvate, or stereoisomer thereof, R¹, R², and R³ are each independently —CH₃ substituted with one R^{a}.

In some embodiments of a compound of Formula (I), or a pharmaceutically acceptable salt, solvate, or stereoisomer thereof, R⁴ is —CH₂CH₃ substituted with two R^{a}.

In some embodiments of a compound of Formula (I), or a pharmaceutically acceptable salt, solvate, or stereoisomer thereof, R¹ is alkyl substituted with a heteroaryl optionally substituted with one of more R^{b}. In some embodiments of a compound of Formula (I), or a pharmaceutically acceptable salt, solvate, or stereoisomer thereof, R¹ is alkyl substituted with an indolyl optionally substituted with one of more R^{b}. In some embodiments of a compound of Formula (I), or a pharmaceutically acceptable salt, solvate, or stereoisomer thereof, R¹ is alkyl substituted with an indolyl optionally substituted with one R^{b}.

In some embodiments of a compound of Formula (I), or a pharmaceutically acceptable salt, solvate, or stereoisomer thereof, R² is alkyl substituted with an aryl optionally substituted with one of more R^{b}. In some embodiments of a compound of Formula (I), or a pharmaceutically acceptable salt, solvate, or stereoisomer thereof, R² is alkyl substituted with a phenyl optionally substituted with one R^{b}. In some embodiments of a compound of Formula (I), or a pharmaceutically acceptable salt, solvate, or stereoisomer thereof, R² is alkyl substituted with a phenyl optionally substituted with one of more R^{b}.

In some embodiments of a compound of Formula (I), or a pharmaceutically acceptable salt, solvate, or stereoisomer thereof, R³ is alkyl substituted with a heteroaryl optionally substituted with one of more R^{b}. In some embodiments of a compound of Formula (I), or a pharmaceutically acceptable salt, solvate, or stereoisomer thereof, R³ is alkyl substituted with a pyridyl optionally substituted with one of more R^{b}. In some embodiments of a compound of Formula (I), or a pharmaceutically acceptable salt, solvate, or stereoisomer thereof, R³ is alkyl substituted with a pyridyl optionally substituted with one R^{b}.

In some embodiments of a compound of Formula (I), or a pharmaceutically acceptable salt, solvate, or stereoisomer thereof, R⁴ is alkyl substituted with a heteroaryl optionally substituted with one of more R^{b}. In some embodiments of a compound of Formula (I), or a pharmaceutically acceptable salt, solvate, or stereoisomer thereof, R⁴ is alkyl substituted with an indolyl optionally substituted with one of more R^{b}. In some embodiments of a compound of Formula (I), or a pharmaceutically acceptable salt, solvate, or stereoisomer thereof, R⁴ is alkyl substituted with R⁶ and with a heteroaryl optionally substituted with one of more R^{b}. In some embodiments of a compound of Formula (I), or a pharmaceutically acceptable salt, solvate, or stereoisomer thereof, R⁴ is alkyl substituted with R⁶ and with an indolyl optionally substituted with one of more R^{b}. In some embodiments of a compound of Formula (I), or a pharmaceutically acceptable salt, solvate, or stereoisomer thereof, R⁴ is alkyl substituted with R⁶ and with an indolyl optionally substituted with one R^{b}.

In some embodiments of a compound of Formula (I), or a pharmaceutically acceptable salt, solvate, or stereoisomer thereof, R^{b} is —H, —OH, —OCH₃, —OC₂H₅, —F, —Cl, —Br, —I, —NH₂, —S(═O)₂NH₂, —CF₃, —C(═O)NH₂, —NHC(═O)H, —NHC(═O)CH₃, —NO₂, —S(═O)CH₃, —NHS(═O)₂CH₃, —OCF₃, —OCH(CH₃)₂, —CH(CH₃)₂, —CH₂CH₂CH₃, —OCH₂CH₂CH₃, —OCH₂OCH₃, or —OCH₂CH₂OCH₃.

In some embodiments of a compound of Formula (I), or a pharmaceutically acceptable salt, solvate, or stereoisomer thereof, R^{b} is —H, —OH, —OCH₃, —F, or —Cl.

In some embodiments of a compound of Formula (I), or a pharmaceutically acceptable salt, solvate, or stereoisomer thereof, R⁶ is —H, —OH, —OCH₃, —OC₂H₅, —F, —Cl, —Br, —I, —NH₂, S(═O)₂NH₂, —CF₃, —C(═O)NH₂, —NHC(═O)H, —NHC(═O)CH₃, —NO₂, —S(═O)CH₃, —NHS(═O)₂CH₃, —OCF₃, —OCH(CH₃)₂, —CH(CH₃)₂, —CH₂CH₂CH₃, —OCH₂CH₂CH₃, —OCH₂OCH₃, —OCH₂CH₂OCH₃, —NHC(═O)H, or —NHC(═O)CH₃. In some embodiments of a compound of Formula (I), or a pharmaceutically acceptable salt, solvate, or stereoisomer thereof, R⁶ is —H or —C(═O)NH₂.

In some embodiments of a compound of Formula (I), or a pharmaceutically acceptable salt, solvate, or stereoisomer thereof, X¹, X², X³, and X⁴ are each independently —C(═O)NH— or —NHC(═O)—. In some embodiments of a compound of Formula (I), or a pharmaceutically acceptable salt, solvate, or stereoisomer thereof, X¹, X², X³, and X⁴ are each —C(═O)NH—.

In some embodiments of a compound of Formula (I), or a pharmaceutically acceptable salt, solvate, or stereoisomer thereof, the compound of Formula (I) has the Formula (Ia):

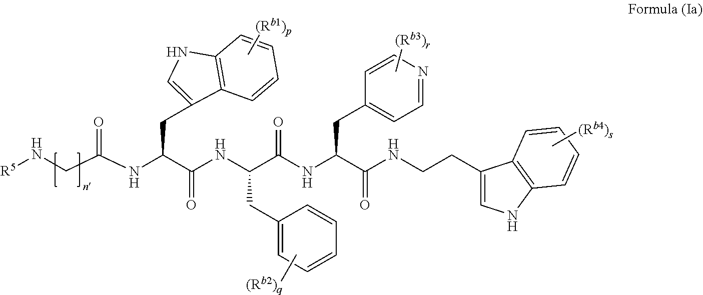

wherein
R⁵ is —H, —CH₃, —C₂H₅, —C(═O)H, —C(═O)aryl, —C(═O)alkyl, or —C(═O)(CH₂CH₂O)ₖCH₃;
R^{b1}, R^{b2}, R^{b3}, and R^{b4} are independently —H, —OH, —OCH₃, —OC₂H₅, —F, —Cl, —Br, —I, —NH₂, —S(═O)₂NH₂, —CF₃, —C(═O)NH₂, —NHC(═O)H, —NHC(═O)CH₃, —NO₂, —S(═O)CH₃, —NHS(═O)₂CH₃, —OCF₃, —OCH(CH₃)₂, —CH(CH₃)₂, —CH₂CH₂CH₃, —OCH₂CH₂CH₃, —OCH₂OCH₃, or —OCH₂CH₂OCH₃;
n′ is 0-4;
k is 1-1000; and
p, q, r, and s are each independently 1-3.

In some embodiments of a compound of Formula (I), or a pharmaceutically acceptable salt, solvate, or stereoisomer thereof, the compound of Formula (I) has the Formula (Ib):

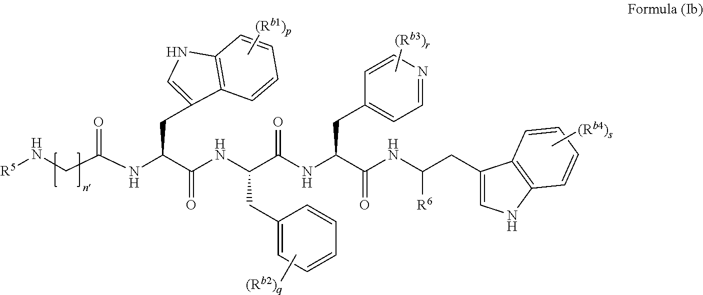

wherein
R⁵ is —H, —CH₃, —C₂H₅, —C(═O)H, —C(═O)aryl, —C(═O)alkyl, or —C(═O)(CH₂CH₂O)ₖCH₃;
R⁶ is —H, —OH, —OCH₃, —OC₂H₅, —F, —Cl, —Br, —I, —NH₂, S(═O)₂NH₂, —CF₃, —C(═O)NH₂, —NHC(═O)H, —NHC(═O)CH₃, —NO₂, —S(═O)CH₃, —NHS(═O)₂CH₃, —OCF₃, —OCH(CH₃)₂, —CH(CH₃)₂, —CH₂CH₂CH₃, —OCH₂CH₂CH₃, —OCH₂OCH₃, —OCH₂CH₂OCH₃, —NHC(═O)H, NHC(═O)CH₃;
R^{b1}, R^{b2}, R^{b3}, and R^{b4} are independently —H, —OH, —OCH₃, —OC₂H₅, —F, —Cl, —Br, —I, —NH₂, —S(═O)₂NH₂, —CF₃, —C(═O)NH₂, —NHC(═O)H, —NHC(═O)CH₃, —NO₂, —S(═O)CH₃, —NHS(═O)₂CH₃, —OCF₃, —OCH(CH₃)₂, —CH(CH₃)₂, —CH₂CH₂CH₃, —OCH₂CH₂CH₃, —OCH₂OCH₃, or —OCH₂CH₂OCH₃;
n′ is 0-4;
k is 1-1000; and
p, q, r, and s are each independently 1-3.

In some embodiments of a compound of Formula (Ib), or a pharmaceutically acceptable salt, solvate, or stereoisomer thereof, R⁶ is —H or —C(═O)NH₂.

In some embodiments of a compound of Formula (Ia) or Formula (Ib), or a pharmaceutically acceptable salt, solvate, or stereoisomer thereof, n′ is 1.

In some embodiments of a compound of Formula (Ia) or Formula (Ib), or a pharmaceutically acceptable salt, solvate, or stereoisomer thereof, n′ is 3.

In some embodiments of a compound of Formula (Ia) or Formula (Ib), or a pharmaceutically acceptable salt, solvate, or stereoisomer thereof, n′ is 4.

In some embodiments of a compound of Formula (Ia) or Formula (Ib), or a pharmaceutically acceptable salt, solvate, or stereoisomer thereof, R^{b1}, R^{b2}, R^{b3}, and R^{b4} are independently —H, —OH, —OCH₃, —F, or —Cl.

In some embodiments of a compound of Formula (Ia) or Formula (Ib), or a pharmaceutically acceptable salt, solvate, or stereoisomer thereof, p is 1; and R^{b1} is —OH.

In some embodiments of a compound of Formula (Ia) or Formula (Ib), or a pharmaceutically acceptable salt, solvate, or stereoisomer thereof, q is 1; and R^{b2} —Cl.

In some embodiments of a compound of Formula (Ia) or Formula (Ib), or a pharmaceutically acceptable salt, solvate, or stereoisomer thereof, r is 1; and R^{b3} is —H.

In some embodiments of a compound of Formula (Ia) or Formula (Ib), or a pharmaceutically acceptable salt, solvate, or stereoisomer thereof, s is 1; and R^{b4} is —H, —OH, —OCH₃, —F, or —Cl.

In some embodiments of a compound of Formula (I), Formula (Ia), or Formula (Ib), or a pharmaceutically acceptable salt, solvate, or stereoisomer thereof, R⁵ is —H. In some embodiments of a compound of Formula (I), Formula (Ia), or Formula (Ib), or a pharmaceutically acceptable salt, solvate, or stereoisomer thereof, R⁵ is —C(═O)(CH₂CH₂O)ₖCH₃.

In some embodiments of a compound of Formula (I), Formula (Ia), or Formula (Ib), or a pharmaceutically acceptable salt, solvate, or stereoisomer thereof, k is 1 to 100. In some embodiments of a compound of Formula (I), Formula (Ia), or Formula (Ib), or a pharmaceutically acceptable salt, solvate, or stereoisomer thereof, k is 100 to 500. In some embodiments of a compound of Formula (I), Formula (Ia), or Formula (Ib), or a pharmaceutically acceptable salt, solvate, or stereoisomer thereof, k is 500 to 1000.

Another aspect provides a pharmaceutical composition comprising a compound of Formula (I), Formula (Ia), or Formula (Ib) and a pharmaceutically acceptable excipient.

Another aspect provides a pharmaceutical composition comprising a compound of Formula (I), Formula (Ia), or Formula (Ib) and a pharmaceutically acceptable excipient, wherein the pharmaceutical composition is in the form of nanoparticles.

Another aspect provides a method of treating amyotrophic lateral sclerosis (ALS) in a subject in need thereof comprising administering to the subject a therapeutically effective amount of a compound of Formula (I), Formula (Ia), or Formula (Ib). Another aspect provides a method of treating amyotrophic lateral sclerosis (ALS) in a subject in need thereof comprising administering to the subject a pharmaceutical composition comprising a compound of Formula (I), Formula (Ia), or Formula (Ib) and a pharmaceutically acceptable excipient. Another aspect provides a method of treating abnormal blood clotting in a subject in need thereof comprising administering to the subject a therapeutically effective amount of a compound of Formula (I), Formula (Ia), or Formula (Ib). A method of treating abnormal blood clotting in a subject in need thereof comprising administering to the subject a pharmaceutical composition comprising a compound of Formula (I), Formula (Ia), or Formula (Ib) and a pharmaceutically acceptable excipient. Another aspect provides a method of treating gastric cancer in a subject in need thereof comprising administering to the subject a therapeutically effective amount of a compound of Formula (I), Formula (Ia), or Formula (Ib). A method of treating gastric cancer in a subject in need thereof comprising administering to the subject a pharmaceutical composition comprising a compound of Formula (I), Formula (Ia), or Formula (Ib) and a pharmaceutically acceptable excipient. Another aspect provides a method of treating spinal cord injury in a subject in need thereof comprising administering to the subject a therapeutically effective amount of a compound of Formula (I), Formula (Ia), or Formula (Ib). A method of treating spinal cord injury in a subject in need thereof comprising administering to the subject a pharmaceutical composition comprising a compound of Formula (I), Formula (Ia), or Formula (Ib) and a pharmaceutically acceptable excipient. Another aspect provides a method of treating Alzheimer's disease (AD) in a subject in need thereof comprising administering to the subject a therapeutically effective amount of a compound of Formula (I), Formula (Ia), or Formula (Ib). A method of treating Alzheimer's disease (AD) in a subject in need thereof comprising administering to the subject a pharmaceutical composition comprising a compound of Formula (I), Formula (Ia), or Formula (Ib) and a pharmaceutically acceptable excipient. Another aspect provides a method of treating traumatic brain injury in a subject in need thereof comprising administering to the subject a therapeutically effective amount of a compound of Formula (I), Formula (Ia), or Formula (Ib). A method of treating traumatic brain injury in a subject in need thereof comprising administering to the subject a pharmaceutical composition comprising a compound of Formula (I), Formula (Ia), or Formula (Ib) and a pharmaceutically acceptable excipient. In some embodiments, the compound of Formula (I), Formula (Ia), or Formula (Ib) or the pharmaceutical composition comprising a compound of Formula (I), Formula (Ia), or Formula (Ib) and a pharmaceutically acceptable excipient is administered intranasally, orally, parenterally, intravenously, intraperitoneally, intramuscularly, topically or subcutaneously.

### BRIEF DESCRIPTION OF THE DRAWINGS

FIG. 1 shows the binding studies with Compound 7 and EphA4. (A) Kᵢ of compound 7 as measured by FPA (fluorescence polarization assay). (B) K_{d} of compound 7 as measured by ITC (isothermal titration calorimetry).

FIG. 2 shows (A) ITC data for the interactions between Compound 7 and the indicated constructs, and (B) ITC data for the interactions between Compound 7 and the EphA2 receptor.

FIG. 3 shows the in vivo efficacy studies with Compound 7. (A) Cumulative probabilities of survival time of SOD1(G93A) mice treated with Compound 7 or saline alone. (B) Average survival time from disease onset to end point and prolonged overall life span in SOD1(G93A) mice treated with Compound 7 or saline alone.

### DETAILED DESCRIPTION OF THE INVENTION

EphA4 belongs to the Eph family of receptor tyrosine kinases, which together with their membrane-bound ligands, the ephrins (Eph receptor-interacting proteins), generate bidirectional signals controlling a multitude of cellular processes during development and in the adult. These receptors possess an extracellular ligand binding domain (LBD) that engages ephrin ligands and intracellular domains including a kinase domain, a Sam domain and PDZ binding motif that initiate the signal transduction cascade. While targeting the kinase domain is a possible avenue to a given Eph receptor inhibition, selectivity is problematic given the highly conserved kinase domain among these receptors and with other kinases. However targeting the ligand binding domain (LBD) of the Eph receptor could lead to more potent and selective inhibitors. The critical roles of EphA4 in other physiological and pathological processes have been reported in recent studies validating EphA4 as a promising target for the development of small molecule drugs to treat several human diseases, including abnormal blood clotting, and spinal cord injury, in addition to amyotrophic lateral sclerosis.

Previous structural studies indicate that the EphA4 LBD contains a hydrophobic pocket surrounded by four flexible loops, which confer large structural plasticity to accommodate different binding partners. Several 12-amino-acid-long peptide binders that selectively block ephrin ligands from binding to EphA4 have been reported. For instance, the APY, KYL, and VTM peptides (which were named based on the first three amino acids of their sequences) bind to EphA4 tightly with Kd values in the low micromolar range. In addition, a few small molecular weight compounds that inhibit ephrin binding to EphA4 at low micromolar concentration have also been reported from HTS campaigns. However, their detailed mechanism of action remains unclear and likely complex, possibly involving compound oxidations or non-specific binding, which are typical issues encountered in traditional HTS hits.

### Definitions

As used in the specification and appended claims, unless specified to the contrary, the following terms have the meaning indicated below.

“Amino” refers to the —NH₂ radical.

“Cyano” or “nitrile” refers to the —CN radical.

“Hydroxy” or “hydroxyl” refers to the —OH radical.

“Nitro” refers to the —NO₂ radical.

“Oxo” refers to the ═O substituent.

“Oxime” refers to the ═N—OH substituent.

“Thioxo” refers to the ═S substituent.

“Alkyl” refers to a linear or branched hydrocarbon chain radical, which is fully saturated, has from one to thirty carbon atoms, and is attached to the rest of the molecule by a single bond. Alkyls comprising any number of carbon atoms from 1 to 30 are included. An alkyl comprising up to 30 carbon atoms is referred to as a C₁-C₃₀ alkyl, likewise, for example, an alkyl comprising up to 12 carbon atoms is a C₁-C₁₂ alkyl. Alkyls (and other moieties defined herein) comprising other numbers of carbon atoms are represented similarly. Alkyl groups include, but are not limited to, C₁-C₃₀ alkyl, C₁-C₂₀ alkyl, C₁-C₁₅ alkyl, C₁-C₁₀ alkyl, C₁-C₈ alkyl, C₁-C₆ alkyl, C₁-C₄ alkyl, C₁-C₃ alkyl, C₁-C₂ alkyl, C₂-C₈ alkyl, C₃-C₈ alkyl, C₄-C₈ alkyl, and C₅-C₁₂ alkyl. Representative alkyl groups include, but are not limited to, methyl, ethyl, n-propyl, 1-methylethyl (isopropyl), n-butyl, i-butyl, s-butyl, n-pentyl, 1,1-dimethylethyl (t-butyl), 2-ethylpropyl, and the like. Representative linear alkyl groups include, but are not limited to, methyl, ethyl, n-propyl, n-butyl, n-pentyl and the like. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, cycloalkyl, cycloalkylalkyl, aryl, aralkyl, heterocycloalkyl, heterocycloalkylalkyl, heteroaryl or heteroarylalkyl, and each R^{f} is independently alkyl, fluoroalkyl, cycloalkyl, cycloalkylalkyl, aryl, aralkyl, heterocycloalkyl, heterocycloalkylalkyl, heteroaryl or heteroarylalkyl.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In certain embodiments, an alkenyl comprises two to six carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a},

—SR^{a}, —OC(O)—R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, cycloalkyl, cycloalkylalkyl, aryl, aralkyl, heterocycloalkyl, heterocycloalkylalkyl, heteroaryl or heteroarylalkyl, and each R^{f} is independently alkyl, fluoroalkyl, cycloalkyl, cycloalkylalkyl, aryl, aralkyl, heterocycloalkyl, heterocycloalkylalkyl, heteroaryl or heteroarylalkyl.

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In certain embodiments, an alkynyl comprises two to six carbon atoms. In other embodiments, an alkynyl has two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo,

trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, cycloalkyl, cycloalkylalkyl, aryl, aralkyl, heterocycloalkyl, heterocycloalkylalkyl, heteroaryl or heteroarylalkyl, and each R^{f} is independently alkyl, fluoroalkyl, cycloalkyl, cycloalkylalkyl, aryl, aralkyl, heterocycloalkyl, heterocycloalkylalkyl, heteroaryl or heteroarylalkyl.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, cycloalkyl, cycloalkylalkyl, aryl, aralkyl, heterocycloalkyl, heterocycloalkylalkyl, heteroaryl or heteroarylalkyl, and each R^{f} is independently alkyl, fluoroalkyl, cycloalkyl, cycloalkylalkyl, aryl, aralkyl, heterocycloalkyl, heterocycloalkylalkyl, heteroaryl or heteroarylalkyl.

“Aminoalkyl” refers to a radical of the formula —R^{c}—N(R^{a})₂ or —R^{c}—N(R^{a})—R^{c}, where each R^{c} is independently an alkylene chain as defined above, for example, methylene, ethylene, and the like; and each R^{a} is independently hydrogen, alkyl, fluoroalkyl, cycloalkyl, cycloalkylalkyl, aryl, aralkyl, heterocycloalkyl, heterocycloalkylalkyl, heteroaryl or heteroarylalkyl.

“Alkoxy” refers to a radical of the formula —OR^{a} where R^{a} is an alkyl radical as defined. Unless stated otherwise specifically in the specification, an alkoxy group may be optionally substituted as described above for alkyl.

“Aryl” refers to a radical derived from a hydrocarbon ring system comprising hydrogen, 6 to 30 carbon atoms and at least one aromatic ring. The aryl radical may be a monocyclic, bicyclic, tricyclic or tetracyclic ring system, which may include fused (when fused with a cycloalkyl or heterocycloalkyl ring, the aryl is bonded through an aromatic ring atom) or bridged ring systems. Aryl radicals include, but are not limited to, aryl radicals derived from the hydrocarbon ring systems of aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group is optionally substituted by one or more of the following substituents: alkyl, alkenyl, alkynyl, halo, fluoroalkyl, cyano, nitro, aryl, aralkyl, aralkenyl, aralkynyl, cycloalkyl, cycloalkylalkyl, heterocycloalkyl, heterocycloalkylalkyl, heteroaryl,

heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2), and —R^{b}—S(O)ₜN(R^{a})₂ (where t is 1 or 2), where each R^{a} is independently hydrogen, alkyl, fluoroalkyl, cycloalkyl, cycloalkylalkyl, aryl (optionally substituted with one or more halo groups), aralkyl, heterocycloalkyl (optionally substituted with one or more alkyl groups), heterocycloalkylalkyl, heteroaryl or heteroarylalkyl, or two R^{a} attached to the same nitrogen atom are combined to form a heterocycloalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aryloxy” refers to a radical bonded through an oxygen atom of the formula —O-aryl, where aryl is as defined above.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Cycloalkyl” or “carbocycle” refers to a stable, non-aromatic, monocyclic or polycyclic carbocyclic ring, which may include fused (when fused with an aryl or a heteroaryl ring, the cycloalkyl is bonded through a non-aromatic ring atom) or bridged ring systems, which is saturated or unsaturated. Representative cycloalkyls or carbocycles include, but are not limited to, cycloalkyls having from three to fifteen carbon atoms, from three to ten carbon atoms, from three to eight carbon atoms, from three to six carbon atoms, from three to five carbon atoms, or three to four carbon atoms. Monocyclic cycloalkyls or carbocycles include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Unless otherwise stated specifically in the specification, the cycloalkyl is optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted heterocycloalkyl, optionally substituted heterocycloalkylalkyl, optionally substituted heteroaryl, optionally substituted

heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocycloalkyl, heterocycloalkylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Cycloalkylalkyl” refers to a radical of the formula —R^{c}-cycloalkyl where R^{c} is an alkylene chain as defined above. The alkylene chain and the cycloalkyl radical are optionally substituted as defined above.

“Fused” refers to any ring structure described herein which is fused to an existing ring structure. When the fused ring is a heretocycloalkyl ring or a heteroaryl ring, any carbon atom on the existing ring structure which becomes part of the fused heretocycloalkyl ring or the fused heteroaryl ring may be replaced with a nitrogen atom.

“Heteroalkyl” refers to a straight or branched hydrocarbon chain alkyl radical containing no unsaturation, having from one to fifteen carbon atoms (e.g., C₁-C₁₅ alkyl) consisting of carbon and hydrogen atoms and one or two heteroatoms selected from O, N, and S, wherein the nitrogen or sulfur atoms may be optionally oxidized and the nitrogen atom may be quaternized. The heteroatom(s) may be placed at any position of the heteroalkyl group including between the rest of the heteroalkyl group and the fragment to which it is attached. The heteroalkyl is attached to the rest of the molecule by a single bond. Unless stated otherwise specifically in the specification, a heteroalkyl group is optionally substituted by one or more of the following substituents: halo, cyano, nitro, oxo, thioxo, imino, oximo,

trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, cycloalkyl, cycloalkylalkyl, aryl, aralkyl, heterocycloalkyl, heterocycloalkylalkyl, heteroaryl or heteroarylalkyl, and each R^{f} is independently alkyl, fluoroalkyl, cycloalkyl, cycloalkylalkyl, aryl, aralkyl, heterocycloalkyl, heterocycloalkylalkyl, heteroaryl or heteroarylalkyl.

“Halo” or “halogen” refers to bromo, chloro, fluoro or iodo. In some embodiments, halogen refers to chloro or fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Haloalkoxy” similarly refers to a radical of the formula —OR^{a} where R^{a} is a haloalkyl radical as defined. Unless stated otherwise specifically in the specification, a haloalkoxy group may be optionally substituted as described below.

“Heterocycloalkyl” or “heterocycle” refers to a stable 3- to 24-membered non-aromatic ring radical comprising 2 to 23 carbon atoms and from one to 8 heteroatoms selected from the group consisting of nitrogen, oxygen, phosphorous and sulfur. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom) or bridged ring systems; and the nitrogen, carbon or sulfur atoms in the heterocycloalkyl radical may be optionally oxidized; the nitrogen atom may be optionally quaternized; and the heterocycloalkyl radical may be partially or fully saturated. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, 2-oxo-1,3-dioxol-4-yl, 1,1-dioxidotetrahydro-2H-thiopyranyl, tetrahydro-2H-thiopyranyl, and tetrahydro-2H-pyranyl. The term heterocycloalkyl also includes all ring forms of the carbohydrates, including but not limited to the monosaccharides, the disaccharides and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group is optionally substituted by one or more of the following substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted heterocycloalkyl, optionally substituted heterocycloalkylalkyl, optionally substituted heteroaryl, optionally substituted

heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocycloalkyl, heterocycloalkylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Heterocycloalkyllalkyl” refers to a radical of the formula —R^{c}-heterocycloalkyl where R^{c} is an alkylene chain as defined above. If the heterocycloalkyl is a nitrogen-containing heterocycloalkyl, the heterocycloalkyl is optionally attached to the alkyl radical at the nitrogen atom. The alkylene chain of the heterocycloalkylslkyl radical is optionally substituted as defined above for an alkylene chain. The heterocycloalkyl part of the heterocycloalkylalkyl radical is optionally substituted as defined above for a heterocycloalkyl group.

“Heterocycloalkylalkoxy” refers to a radical bonded through an oxygen atom of the formula —O—R^{c}-heterocycloalkyl where R^{c} is an alkylene chain as defined above. If the heterocycloalkyl is a nitrogen-containing heterocycloalkyl, the heterocycloalkyl is optionally attached to the alkyl radical at the nitrogen atom. The alkylene chain of the heterocycloalkylalkoxy radical is optionally substituted as defined above for an alkylene chain. The heterocycloalkyl part of the heterocycloalkylalkoxy radical is optionally substituted as defined above for a heterocycloalkyl group.

“Heteroaryl” refers to a 5- to 14-membered ring system radical comprising hydrogen atoms, one to thirteen carbon atoms, one to six heteroatoms selected from the group consisting of nitrogen, oxygen, phosphorous and sulfur, and at least one aromatic ring. In some embodiments, the heteroaryl is a 5-membered heteroaryl. In some embodiments, the heteroaryl is a 6-membered heteroaryl. For purposes of this invention, the heteroaryl radical may be a monocyclic, bicyclic, tricyclic or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group is optionally substituted by one or more of the following substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted heterocycloalkyl, optionally substituted heterocycloalkylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocycloalkyl, heterocycloalkylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroaryloxy” refers to radical bonded through an oxygen atom of the formula —O— heteroaryl, where heteroaryl is as defined above.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

A “tautomer” refers to a molecule wherein a proton shift from one atom of a molecule to another atom of the same molecule is possible. In certain embodiments, the compounds presented herein exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

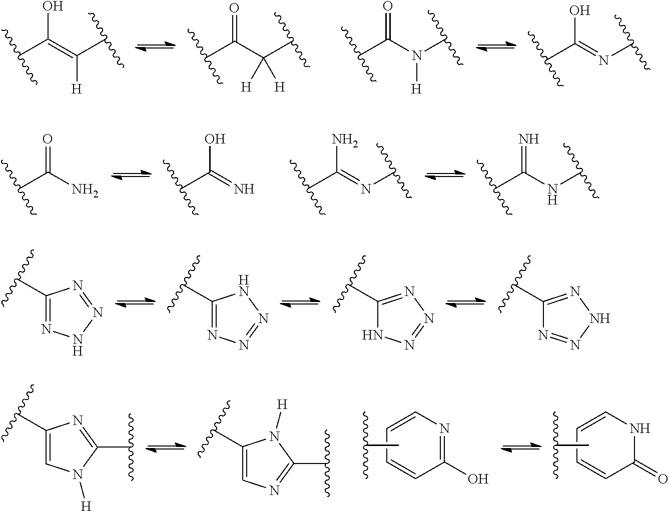

“Optional” or “optionally” means that a subsequently described event or circumstance may or may not occur and that the description includes instances when the event or circumstance occurs and instances in which it does not. For example, “optionally substituted aryl” means that the aryl radical may or may not be substituted and that the description includes both substituted aryl radicals and aryl radicals having no substitution. “Optionally substituted” and “substituted or unsubstituted” and “unsubstituted or substituted” are used interchangeably herein.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are prepared by contacting the free base forms with a sufficient amount of the desired acid to produce the salt.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. In some embodiments, pharmaceutically acceptable base addition salts are formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof.

When ranges are used herein for physical properties, such as molecular weight, or chemical properties, such as chemical formulae, all combinations and subcombinations of ranges and specific embodiments therein are intended to be included.

The term “about” when referring to a number or a numerical range means that the number or numerical range referred to is an approximation within experimental variability (or within statistical experimental error), and thus the number or numerical range varies between 1% and 15% of the stated number or numerical range.

The term “comprising” (and related terms such as “comprise” or “comprises” or “having” or “including”) is not intended to exclude that which in other certain embodiments, for example, an embodiment of any composition of matter, composition, method, or process, or the like, described herein, “consist of” or “consist essentially of” the described features.

The term “subject” or “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

As used herein, “treatment” or “treating” or “palliating” or “ameliorating” are used interchangeably herein. These terms refers to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are administered to a patient at risk of developing a particular disease, or to a patient reporting one or more of the physiological symptoms of a disease, even though a diagnosis of this disease has been made.

### Compounds

Compounds described herein are EphA4 inhibitors. In some embodiments, the compounds described herein inhibit EphA4 by targeting its ligand binding domain. These compounds, and compositions comprising these compounds, are useful for the treatment of diseases or conditions where the EphA4 receptor is involved including, but not limited to ALS, abnormal blood clotting, gastric cancer, spinal cord injury, Alzheimer's disease, or traumatic brain injury. In some embodiments, these compounds, and compositions comprising these compounds, are useful for the treatment of ALS.

One aspect provides a compound of formula (I), or a pharmaceutically acceptable salt, solvate, or stereoisomer thereof:

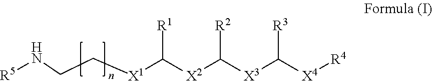

wherein
X¹, X², X³, and X⁴ are each independently —C(═O)NH—, —NHC(═O)—, —S(═O)₂NH—, —O—, —CH₂—, —CH₂CH₂—, —OCH₂—, —CH₂O—, —S—, —S(═O)₂—, or —C(═O)NHS(═O)₂—;
R¹, R², R³, and R⁴ are each independently alkyl, heterocycloalkyl, or cycloalkyl; wherein the alkyl, heterocycloalkyl, and cycloalkyl are optionally substituted with one of more R^{a};
R⁵ is —H, —CH₃, —C₂H₅, —C(═O)H, —C(═O)aryl, —C(═O)alkyl, or —C(═O)(CH₂CH₂O)ₖCH₃;
R⁶ is —H, halogen, alkyl, haloalkyl, alkoxy, haloalkoxy, —OH, —NH₂, S(═O)₂NH₂, —C(═O)NH₂, —NHC(═O)H, —NHC(═O)CH₃, —NO₂, —S(═O)CH₃, —NHS(═O)₂CH₃, —OCH₂OCH₃, —OCH₂CH₂OCH₃, —NHC(═O)H, or —NHC(═O)CH₃;
R^{a} is R⁶, aryl or heteroaryl; wherein the aryl or heteroaryl are optionally substituted with one or more R^{b};
R^{b} is —H, halogen, alkyl, haloalkyl, alkoxy, haloalkoxy, —OH, —NH₂, —S(═O)₂NH₂, —C(═O)NH₂, —NHC(═O)H, —NHC(═O)CH₃, —NO₂, —S(═O)CH₃, —NHS(═O)₂CH₃, —OCH₂OCH₃, or —OCH₂CH₂OCH₃;
n is 0-4; and
k is 1-1000.

In some embodiments of a compound of Formula (I), or a pharmaceutically acceptable salt, solvate, or stereoisomer thereof, n is 2-4. In some embodiments of a compound of Formula (I), or a pharmaceutically acceptable salt, solvate, or stereoisomer thereof, n is 2. In some embodiments of a compound of Formula (I), or a pharmaceutically acceptable salt, solvate, or stereoisomer thereof, n is 3. In some embodiments of a compound of Formula (I), or a pharmaceutically acceptable salt, solvate, or stereoisomer thereof, n is 4. In some embodiments of a compound of Formula (I), or a pharmaceutically acceptable salt, solvate, or stereoisomer thereof, n is not 1. In some embodiments of a compound of Formula (I), or a pharmaceutically acceptable salt, solvate, or stereoisomer thereof, n is not 1 when R⁵ is —H.

In some embodiments of a compound of Formula (I), or a pharmaceutically acceptable salt, solvate, or stereoisomer thereof, R¹, R², R³, and R⁴ are each independently alkyl substituted with one of more R^{a}.

In some embodiments of a compound of Formula (I), or a pharmaceutically acceptable salt, solvate, or stereoisomer thereof, R¹, R², R³, and R⁴ are each independently —CH₃ or —CH₂CH₃ substituted with one of more R^{a}.

In some embodiments of a compound of Formula (I), or a pharmaceutically acceptable salt, solvate, or stereoisomer thereof, R¹, R², R³, and R⁴ are each independently —CH₃ or —CH₂CH₃ substituted with one R^{a}.

In some embodiments of a compound of Formula (I), or a pharmaceutically acceptable salt, solvate, or stereoisomer thereof, R¹, R², R³, and R⁴ are each independently —CH₃ or —CH₂CH₃ substituted with two R^{a}.

In some embodiments of a compound of Formula (I), or a pharmaceutically acceptable salt, solvate, or stereoisomer thereof, R¹, R², and R³ are each independently —CH₃ substituted with one R^{a}.

In some embodiments of a compound of Formula (I), or a pharmaceutically acceptable salt, solvate, or stereoisomer thereof, R⁴ is —CH₂CH₃ substituted with two R^{a}.

In some embodiments of a compound of Formula (I), or a pharmaceutically acceptable salt, solvate, or stereoisomer thereof, R¹ is alkyl substituted with a heteroaryl optionally substituted with one of more R^{b}.

In some embodiments of a compound of Formula (I), or a pharmaceutically acceptable salt, solvate, or stereoisomer thereof, R¹ is alkyl substituted with an indolyl optionally substituted with one of more R^{b}. In some embodiments of a compound of Formula (I), or a pharmaceutically acceptable salt, solvate, or stereoisomer thereof, R¹ is alkyl substituted with 1H-indol-3-yl or 1H-indol-2-yl, each optionally substituted with one of more R^{b}. In some embodiments of a compound of Formula (I), or a pharmaceutically acceptable salt, solvate, or stereoisomer thereof, R¹ is alkyl substituted with 1H-indol-3-yl optionally substituted with one of more R^{b}.

In some embodiments of a compound of Formula (I), or a pharmaceutically acceptable salt, solvate, or stereoisomer thereof, R¹ is alkyl substituted with an indolyl optionally substituted with one R^{b}. In some embodiments of a compound of Formula (I), or a pharmaceutically acceptable salt, solvate, or stereoisomer thereof, R¹ is alkyl substituted with 1H-indol-3-yl or 1H-indol-2-yl, each optionally substituted with one R^{b}. In some embodiments of a compound of Formula (I), or a pharmaceutically acceptable salt, solvate, or stereoisomer thereof, R¹ is alkyl substituted with 1H-indol-3-yl optionally substituted with one R^{b}.

In some embodiments of a compound of Formula (I), or a pharmaceutically acceptable salt, solvate, or stereoisomer thereof, R² is alkyl substituted with an aryl optionally substituted with one of more R^{b}. In some embodiments of a compound of Formula (I), or a pharmaceutically acceptable salt, solvate, or stereoisomer thereof, R² is alkyl substituted with a phenyl optionally substituted with one R^{b}. In some embodiments of a compound of Formula (I), or a pharmaceutically acceptable salt, solvate, or stereoisomer thereof, R² is alkyl substituted with a phenyl optionally substituted with one of more R^{b}.

In some embodiments of a compound of Formula (I), or a pharmaceutically acceptable salt, solvate, or stereoisomer thereof, R³ is alkyl substituted with a heteroaryl optionally substituted with one of more R^{b}.

In some embodiments of a compound of Formula (I), or a pharmaceutically acceptable salt, solvate, or stereoisomer thereof, R³ is alkyl substituted with a pyridyl optionally substituted with one of more R^{b}. In some embodiments of a compound of Formula (I), or a pharmaceutically acceptable salt, solvate, or stereoisomer thereof, R³ is alkyl substituted with 2-pyridyl, 3-pyridyl, or 4-pyridyl, each optionally substituted with one of more R^{b}. In some embodiments of a compound of Formula (I), or a pharmaceutically acceptable salt, solvate, or stereoisomer thereof, R³ is alkyl substituted with 4-pyridyl optionally substituted with one of more R^{b}.

In some embodiments of a compound of Formula (I), or a pharmaceutically acceptable salt, solvate, or stereoisomer thereof, R³ is alkyl substituted with a pyridyl optionally substituted with one R^{b}. In some embodiments of a compound of Formula (I), or a pharmaceutically acceptable salt, solvate, or stereoisomer thereof, R³ is alkyl substituted with 2-pyridyl, 3-pyridyl, or 4-pyridyl, each optionally substituted with one R^{b}. In some embodiments of a compound of Formula (I), or a pharmaceutically acceptable salt, solvate, or stereoisomer thereof, R³ is alkyl substituted with 4-pyridyl optionally substituted with one R^{b}.

In some embodiments of a compound of Formula (I), or a pharmaceutically acceptable salt, solvate, or stereoisomer thereof, R³ is alkyl substituted with an unsubstituted pyridyl. In some embodiments of a compound of Formula (I), or a pharmaceutically acceptable salt, solvate, or stereoisomer thereof, R³ is alkyl substituted with unsubstituted 2-pyridyl, unsubstituted 3-pyridyl, or unsubstituted 4-pyridyl. In some embodiments of a compound of Formula (I), or a pharmaceutically acceptable salt, solvate, or stereoisomer thereof, R³ is alkyl substituted with unsubstituted 4-pyridyl.

In some embodiments of a compound of Formula (I), or a pharmaceutically acceptable salt, solvate, or stereoisomer thereof, R⁴ is alkyl substituted with a heteroaryl optionally substituted with one of more R^{b}.

In some embodiments of a compound of Formula (I), or a pharmaceutically acceptable salt, solvate, or stereoisomer thereof, R⁴ is alkyl substituted with an indolyl optionally substituted with one of more R^{b}. In some embodiments of a compound of Formula (I), or a pharmaceutically acceptable salt, solvate, or stereoisomer thereof, R⁴ is alkyl substituted with 1H-indol-3-yl or 1H-indol-2-yl, each optionally substituted with one of more R^{b}. In some embodiments of a compound of Formula (I), or a pharmaceutically acceptable salt, solvate, or stereoisomer thereof, R⁴ is alkyl substituted with 1H-indol-3-yl optionally substituted with one of more R^{b}.

In some embodiments of a compound of Formula (I), or a pharmaceutically acceptable salt, solvate, or stereoisomer thereof, R⁴ is alkyl substituted with R⁶ and with a heteroaryl optionally substituted with one of more R^{b}.

In some embodiments of a compound of Formula (I), or a pharmaceutically acceptable salt, solvate, or stereoisomer thereof, R⁴ is alkyl substituted with R⁶ and with an indolyl optionally substituted with one of more R^{b}. In some embodiments of a compound of Formula (I), or a pharmaceutically acceptable salt, solvate, or stereoisomer thereof, R⁴ is alkyl substituted with R⁶ and with 1H-indol-3-yl or 1H-indol-2-yl, each optionally substituted with one of more R^{b}. In some embodiments of a compound of Formula (I), or a pharmaceutically acceptable salt, solvate, or stereoisomer thereof, R⁴ is alkyl substituted with R⁶ and with 1H-indol-3-yl optionally substituted with one of more R^{b}. In some embodiments of a compound of Formula (I), or a pharmaceutically acceptable salt, solvate, or stereoisomer thereof, R⁶ is not —H.

In some embodiments of a compound of Formula (I), or a pharmaceutically acceptable salt, solvate, or stereoisomer thereof, R⁴ is alkyl substituted with R⁶ and with an indolyl optionally substituted with one R^{b}. In some embodiments of a compound of Formula (I), or a pharmaceutically acceptable salt, solvate, or stereoisomer thereof, R⁴ is alkyl substituted with R⁶ and with 1H-indol-3-yl or 1H-indol-2-yl, each optionally substituted with one R^{b}. In some embodiments of a compound of Formula (I), or a pharmaceutically acceptable salt, solvate, or stereoisomer thereof, R⁴ is alkyl substituted with R⁶ and with 1H-indol-3-yl optionally substituted with one R^{b}.

In some embodiments of a compound of Formula (I), or a pharmaceutically acceptable salt, solvate, or stereoisomer thereof, R^{b} is —H, —OH, —OCH₃, —OC₂H₅, —F, —Cl, —Br, —I, —NH₂, —S(═O)₂NH₂, —CF₃, —C(═O)NH₂, —NHC(═O)H, —NHC(═O)CH₃, —NO₂, —S(═O)CH₃, —NHS(═O)₂CH₃, —OCF₃, —OCH(CH₃)₂, —CH(CH₃)₂, —CH₂CH₂CH₃, —OCH₂CH₂CH₃, —OCH₂OCH₃, or —OCH₂CH₂OCH₃.

In some embodiments of a compound of Formula (I), or a pharmaceutically acceptable salt, solvate, or stereoisomer thereof, R^{b} is —H, —OH, —OCH₃, —F, or —Cl.

In some embodiments of a compound of Formula (I), or a pharmaceutically acceptable salt, solvate, or stereoisomer thereof, R⁶ is —H, —OH, —OCH₃, —OC₂H₅, —F, —Cl, —Br, —I, —NH₂, S(═O)₂NH₂, —CF₃, —C(═O)NH₂, —NHC(═O)H, —NHC(═O)CH₃, —NO₂, —S(═O)CH₃, —NHS(═O)₂CH₃, —OCF₃, —OCH(CH₃)₂, —CH(CH₃)₂, —CH₂CH₂CH₃, —OCH₂CH₂CH₃, —OCH₂OCH₃, —OCH₂CH₂OCH₃, —NHC(═O)H, or —NHC(═O)CH₃.

In some embodiments of a compound of Formula (I), or a pharmaceutically acceptable salt, solvate, or stereoisomer thereof, R⁶ is —H or —C(═O)NH₂.

In some embodiments of a compound of Formula (I), or a pharmaceutically acceptable salt, solvate, or stereoisomer thereof, X¹, X², X³, and X⁴ are each independently —C(═O)NH— or —NHC(═O)—.

In some embodiments of a compound of Formula (I), or a pharmaceutically acceptable salt, solvate, or stereoisomer thereof, X¹, X², X³, and X⁴ are each —C(═O)NH—.

In some embodiments of a compound of Formula (I), or a pharmaceutically acceptable salt, solvate, or stereoisomer thereof, the compound of Formula (I) has the Formula (Ia):

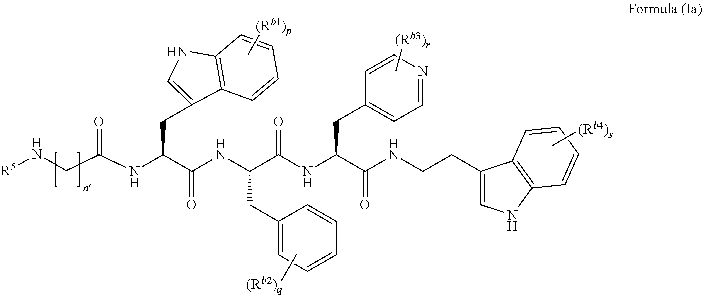

wherein
R⁵ is —H, —CH₃, —C₂H₅, —C(═O)H, —C(═O)aryl, —C(═O)alkyl, or —C(═O)(CH₂CH₂O)ₖCH₃;
R^{b1}, R^{b2}, R^{b3}, and R^{b4} are independently —H, —OH, —OCH₃, —OC₂H₅, —F, —Cl, —Br, —I, —NH₂, —S(═O)₂NH₂, —CF₃, —C(═O)NH₂, —NHC(═O)H, —NHC(═O)CH₃, —NO₂, —S(═O)CH₃, —NHS(═O)₂CH₃, —OCF₃, —OCH(CH₃)₂, —CH(CH₃)₂, —CH₂CH₂CH₃, —OCH₂CH₂CH₃, —OCH₂OCH₃, or —OCH₂CH₂OCH₃;
n′ is 0-4;
k is 1-1000; and
p, q, r, and s are each independently 1-3.

In some embodiments of a compound of Formula (I), or a pharmaceutically acceptable salt, solvate, or stereoisomer thereof, the compound of Formula (I) has the Formula (Ib):

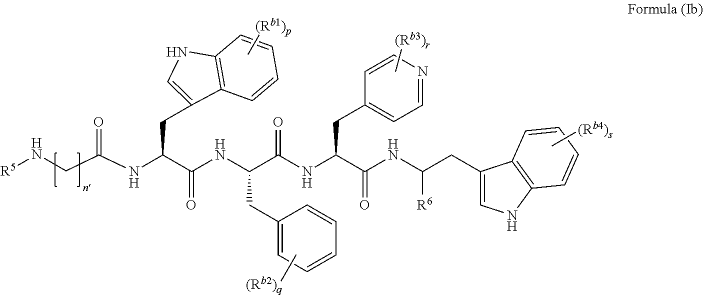

wherein
R⁵ is —H, —CH₃, —C₂H₅, —C(═O)H, —C(═O)aryl, —C(═O)alkyl, or —C(═O)(CH₂CH₂O)ₖCH₃;
R⁶ is —H, —OH, —OCH₃, —OC₂H₅, —F, —Cl, —Br, —I, —NH₂, S(═O)₂NH₂, —CF₃, —C(═O)NH₂, —NHC(═O)H, —NHC(═O)CH₃, —NO₂, —S(═O)CH₃, —NHS(═O)₂CH₃, —OCF₃, —OCH(CH₃)₂, —CH(CH₃)₂, —CH₂CH₂CH₃, —OCH₂CH₂CH₃, —OCH₂OCH₃, —OCH₂CH₂OCH₃, —NHC(═O)H, or —NHC(═O)CH₃;
R^{b1}, R^{b2}, R^{b3}, and R^{b4} are independently —H, —OH, —OCH₃, —OC₂H₅, —F, —Cl, —Br, —I, —NH₂, —S(═O)₂NH₂, —CF₃, —C(═O)NH₂, —NHC(═O)H, —NHC(═O)CH₃, —NO₂, —S(═O)CH₃, —NHS(═O)₂CH₃, —OCF₃, —OCH(CH₃)₂, —CH(CH₃)₂, —CH₂CH₂CH₃, —OCH₂CH₂CH₃, —OCH₂OCH₃, or —OCH₂CH₂OCH₃;
n′ is 0-4;
k is 1-1000; and
p, q, r, and s are each independently 1-3.

In some embodiments of a compound of Formula (Ib), or a pharmaceutically acceptable salt, solvate, or stereoisomer thereof, R⁶ is —H or —C(═O)NH₂.

In some embodiments of a compound of Formula (Ia) or Formula (Ib), or a pharmaceutically acceptable salt, solvate, or stereoisomer thereof, n′ is 1. In some embodiments of a compound of Formula (Ia) or Formula (Ib), or a pharmaceutically acceptable salt, solvate, or stereoisomer thereof, n′ is 3. In some embodiments of a compound of Formula (Ia) or Formula (Ib), or a pharmaceutically acceptable salt, solvate, or stereoisomer thereof, n′ is 4. In some embodiments of a compound of Formula (Ia) or Formula (Ib), or a pharmaceutically acceptable salt, solvate, or stereoisomer thereof, n′ is not 2. In some embodiments of a compound of Formula (Ia) or Formula (Ib), or a pharmaceutically acceptable salt, solvate, or stereoisomer thereof, n′ is not 2 when R⁵ is —H. In some embodiments of a compound of Formula (Ia) or Formula (Ib), or a pharmaceutically acceptable salt, solvate, or stereoisomer thereof, n′ is 2 and R⁶ is not —C(═O)NH₂.

In some embodiments of a compound of Formula (Ia) or Formula (Ib), or a pharmaceutically acceptable salt, solvate, or stereoisomer thereof, R^{b1}, R^{b2}, R^{b3}, and R^{b4} are independently —H, —OH, —OCH₃, —F, or —Cl.

In some embodiments of a compound of Formula (Ia) or Formula (Ib), or a pharmaceutically acceptable salt, solvate, or stereoisomer thereof, p is 0. In some embodiments of a compound of Formula (Ia) or Formula (Ib), or a pharmaceutically acceptable salt, solvate, or stereoisomer thereof, p is 1. In some embodiments of a compound of Formula (Ia) or Formula (Ib), or a pharmaceutically acceptable salt, solvate, or stereoisomer thereof, p is 2. In some embodiments of a compound of Formula (Ia) or Formula (Ib), or a pharmaceutically acceptable salt, solvate, or stereoisomer thereof, p is 3. In some embodiments of a compound of Formula (Ia) or Formula (Ib), or a pharmaceutically acceptable salt, solvate, or stereoisomer thereof, p is 1; and R^{b1} is —OH.

In some embodiments of a compound of Formula (Ia) or Formula (Ib), or a pharmaceutically acceptable salt, solvate, or stereoisomer thereof, q is 0. In some embodiments of a compound of Formula (Ia) or Formula (Ib), or a pharmaceutically acceptable salt, solvate, or stereoisomer thereof, q is 1. In some embodiments of a compound of Formula (Ia) or Formula (Ib), or a pharmaceutically acceptable salt, solvate, or stereoisomer thereof, q is 2. In some embodiments of a compound of Formula (Ia) or Formula (Ib), or a pharmaceutically acceptable salt, solvate, or stereoisomer thereof, q is 3. In some embodiments of a compound of Formula (Ia) or Formula (Ib), or a pharmaceutically acceptable salt, solvate, or stereoisomer thereof, q is 1; and R^{b2} —Cl.

In some embodiments of a compound of Formula (Ia) or Formula (Ib), or a pharmaceutically acceptable salt, solvate, or stereoisomer thereof, r is 0. In some embodiments of a compound of Formula (Ia) or Formula (Ib), or a pharmaceutically acceptable salt, solvate, or stereoisomer thereof, r is 1. In some embodiments of a compound of Formula (Ia) or Formula (Ib), or a pharmaceutically acceptable salt, solvate, or stereoisomer thereof, r is 2. In some embodiments of a compound of Formula (Ia) or Formula (Ib), or a pharmaceutically acceptable salt, solvate, or stereoisomer thereof, r is 3. In some embodiments of a compound of Formula (Ia) or Formula (Ib), or a pharmaceutically acceptable salt, solvate, or stereoisomer thereof, r is 1; and R^{b3} is —H.

In some embodiments of a compound of Formula (Ia) or Formula (Ib), or a pharmaceutically acceptable salt, solvate, or stereoisomer thereof, s is 0. In some embodiments of a compound of Formula (Ia) or Formula (Ib), or a pharmaceutically acceptable salt, solvate, or stereoisomer thereof, s is 1. In some embodiments of a compound of Formula (Ia) or Formula (Ib), or a pharmaceutically acceptable salt, solvate, or stereoisomer thereof, s is 2. In some embodiments of a compound of Formula (Ia) or Formula (Ib), or a pharmaceutically acceptable salt, solvate, or stereoisomer thereof, s is 3. In some embodiments of a compound of Formula (Ia) or Formula (Ib), or a pharmaceutically acceptable salt, solvate, or stereoisomer thereof, s is 1; and R^{b4} is —H, —OH, —OCH₃, —F, or —Cl.

In some embodiments of a compound of Formula (I), Formula (Ia), or Formula (Ib), or a pharmaceutically acceptable salt, solvate, or stereoisomer thereof, R⁵ is —H. In some embodiments of a compound of Formula (I), Formula (Ia), or Formula (Ib), or a pharmaceutically acceptable salt, solvate, or stereoisomer thereof, R⁵ is —C(═O)(CH₂CH₂O)ₖCH₃. In some embodiments of a compound of Formula (I), Formula (Ia), or Formula (Ib), or a pharmaceutically acceptable salt, solvate, or stereoisomer thereof, k is 1 to 100. In some embodiments of a compound of Formula (I), Formula (Ia), or Formula (Ib), or a pharmaceutically acceptable salt, solvate, or stereoisomer thereof, k is 100 to 500. In some embodiments of a compound of Formula (I), Formula (Ia), or Formula (Ib), or a pharmaceutically acceptable salt, solvate, or stereoisomer thereof, k is 500 to 1000. In some embodiments of a compound of Formula (I), Formula (Ia), or Formula (Ib), or a pharmaceutically acceptable salt, solvate, or stereoisomer thereof, k is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100. In some embodiments of a compound of Formula (I), Formula (Ia), or Formula (Ib), or a pharmaceutically acceptable salt, solvate, or stereoisomer thereof, k is at least 10, at least 20, at least 30, at least 40, at least 50, at least 60, at least 70, at least 80, at least 90, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, or at least 1000.

In some embodiments of a compound of Formula (I), or a pharmaceutically acceptable salt, solvate, or stereoisomer thereof, the compound is represented by the following formula:

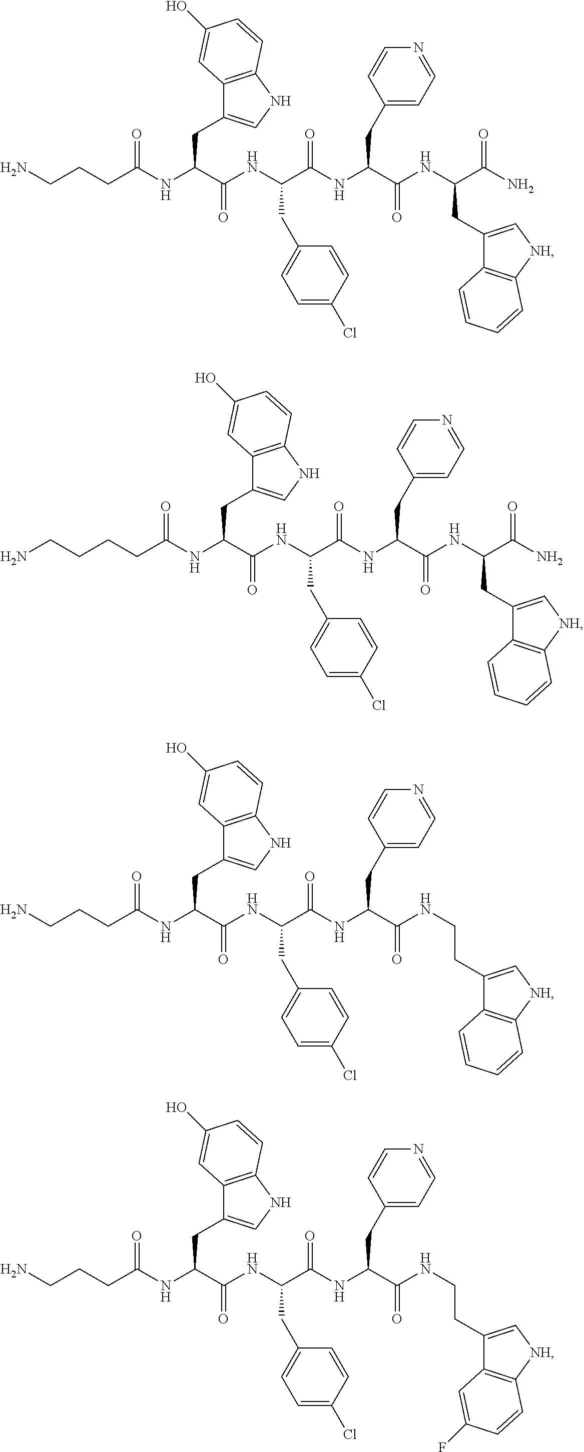 [SMILES: NCCCC(=O)N[C@@H](Cc1c[nH]c2ccc(O)cc12)C(=O)N[C@@H](Cc1ccc(Cl)cc1)C(=O)N[C@@H](Cc1ccncc1)C(=O)N[C@H](Cc1c[nH]c2ccccc12)C(N)=O ; NCCCCC(=O)N[C@@H](Cc1c[nH]c2ccc(O)cc12)C(=O)N[C@@H](Cc1ccc(Cl)cc1)C(=O)N[C@@H](Cc1ccncc1)C(=O)N[C@H](Cc1c[nH]c2ccccc12)C(N)=O ; NCCCC(=O)N[C@@H](Cc1c[nH]c2ccc(O)cc12)C(=O)N[C@@H](Cc1ccc(Cl)cc1)C(=O)N[C@@H](Cc1ccncc1)C(=O)NCCc1c[nH]c2ccccc12 ; NCCCC(=O)N[C@@H](Cc1c[nH]c2ccc(O)cc12)C(=O)N[C@@H](Cc1ccc(Cl)cc1)C(=O)N[C@@H](Cc1ccncc1)C(=O)NCCc1c[nH]c2ccc(F)cc12]

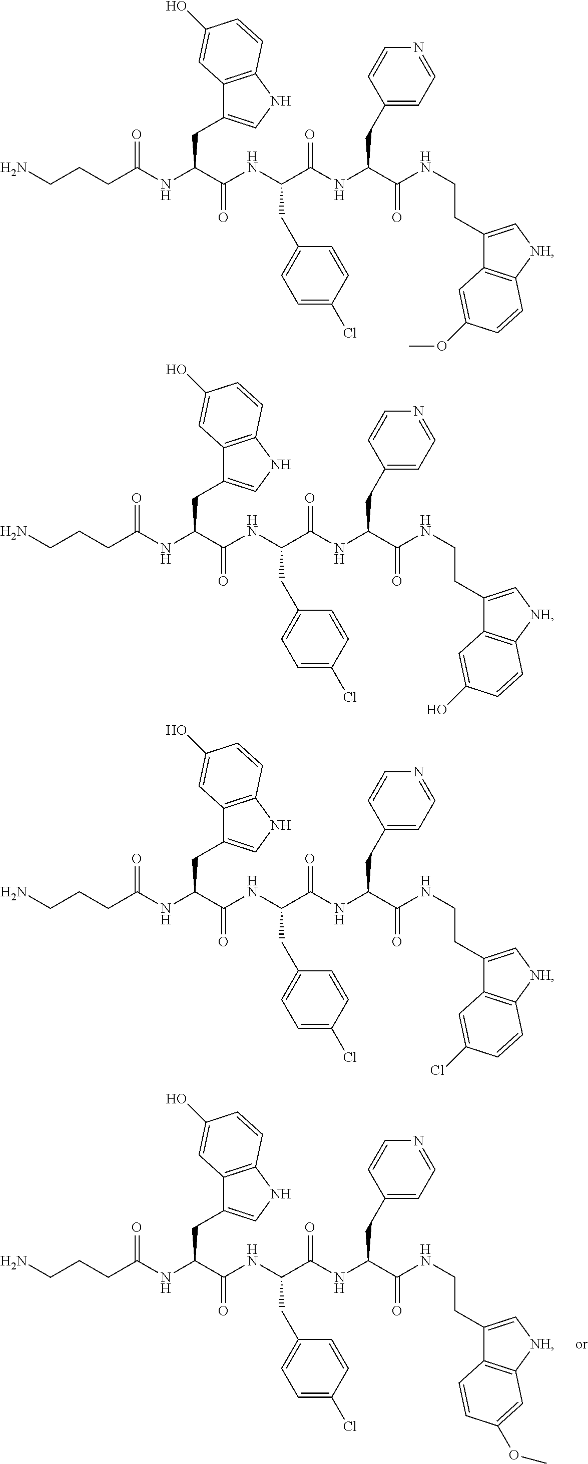 [SMILES: COc1ccc2[nH]cc(CCNC(=O)[C@H](Cc3ccncc3)NC(=O)[C@H](Cc3ccc(Cl)cc3)NC(=O)[C@H](Cc3c[nH]c4ccc(O)cc34)NC(=O)CCCN)c2c1 ; NCCCC(=O)N[C@@H](Cc1c[nH]c2ccc(O)cc12)C(=O)N[C@@H](Cc1ccc(Cl)cc1)C(=O)N[C@@H](Cc1ccncc1)C(=O)NCCc1c[nH]c2ccc(O)cc12 ; NCCCC(=O)N[C@@H](Cc1c[nH]c2ccc(O)cc12)C(=O)N[C@@H](Cc1ccc(Cl)cc1)C(=O)N[C@@H](Cc1ccncc1)C(=O)NCCc1c[nH]c2ccc(Cl)cc12 ; COc1ccc2c(CCNC(=O)[C@H](Cc3ccncc3)NC(=O)[C@H](Cc3ccc(Cl)cc3)NC(=O)[C@H](Cc3c[nH]c4ccc(O)cc34)NC(=O)CCCN)c[nH]c2c1]

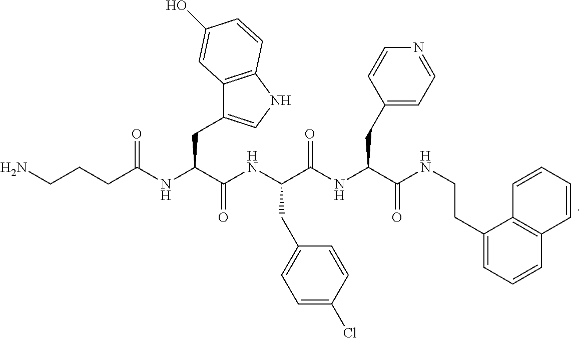 [SMILES: NCCCC(=O)N[C@@H](Cc1c[nH]c2ccc(O)cc12)C(=O)N[C@@H](Cc1ccc(Cl)cc1)C(=O)N[C@@H](Cc1ccncc1)C(=O)NCCc1cccc2ccccc12]

Another aspect provides a compound of formula (II), or a pharmaceutically acceptable salt, solvate, or stereoisomer thereof:

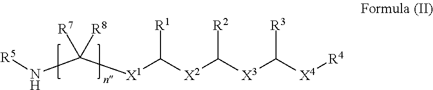

wherein
X¹, X², X³, and X⁴ are each independently —C(═O)NH—, —NHC(═O)—, —S(═O)₂NH—, —O—, —CH₂—, —CH₂CH₂—, —OCH₂—, —CH₂O—, —S—, —S(═O)₂—, or —C(═O)NHS(═O)₂—;
R¹, R², R³, and R⁴ are each independently alkyl, heterocycloalkyl, or cycloalkyl; wherein the alkyl, heterocycloalkyl, and cycloalkyl are optionally substituted with one of more R^{a};
R⁵ is —H, —CH₃, —C₂H₅, —C(═O)H, —C(═O)aryl, —C(═O)alkyl, or —C(═O)(CH₂CH₂O)ₖCH₃;
R⁶ is —H, halogen, alkyl, haloalkyl, alkoxy, haloalkoxy, —OH, —NH₂, S(═O)₂NH₂, —C(═O)NH₂, —NHC(═O)H, —NHC(═O)CH₃, —NO₂, —S(═O)CH₃, —NHS(═O)₂CH₃, —OCH₂OCH₃, —OCH₂CH₂OCH₃, —NHC(═O)H, or —NHC(═O)CH₃;
R⁷ and R⁸ are each independently —H, alkyl, alkoxy, haloalkoxy, halogen, heterocycloalkyl, or cycloalkyl; provided that at least one of R⁷ or R⁸ is not —H;
R^{a} is R⁶, aryl or heteroaryl; wherein the aryl or heteroaryl are optionally substituted with one or more R^{b};
R^{b} is —H, halogen, alkyl, haloalkyl, alkoxy, haloalkoxy, —OH, —NH₂, —S(═O)₂NH₂, —C(═O)NH₂, —NHC(═O)H, —NHC(═O)CH₃, —NO₂, —S(═O)CH₃, —NHS(═O)₂CH₃, —OCH₂OCH₃, or —OCH₂CH₂OCH₃;
n″ is 1-4; and
k is 1-1000.

In some embodiments of a compound of Formula (II), or a pharmaceutically acceptable salt, solvate, or stereoisomer thereof, n″ is 1. In some embodiments of a compound of Formula (II), or a pharmaceutically acceptable salt, solvate, or stereoisomer thereof, n″ is 2. In some embodiments of a compound of Formula (II), or a pharmaceutically acceptable salt, solvate, or stereoisomer thereof, n″ is 3. In some embodiments of a compound of Formula (II), or a pharmaceutically acceptable salt, solvate, or stereoisomer thereof, n″ is 4.

In some embodiments of a compound of Formula (II), or a pharmaceutically acceptable salt, solvate, or stereoisomer thereof, R⁷ and R⁸ are each independently alkyl. In some embodiments of a compound of Formula (II), or a pharmaceutically acceptable salt, solvate, or stereoisomer thereof, R⁷ and R⁸ are each —CH₃.

In some embodiments of a compound of Formula (II), or a pharmaceutically acceptable salt, solvate, or stereoisomer thereof, R¹, R², R³, and R⁴ are each independently alkyl substituted with one of more R^{a}.

In some embodiments of a compound of Formula (II), or a pharmaceutically acceptable salt, solvate, or stereoisomer thereof, R¹, R², R³, and R⁴ are each independently —CH₃ or —CH₂CH₃ substituted with one of more R^{a}.

In some embodiments of a compound of Formula (II), or a pharmaceutically acceptable salt, solvate, or stereoisomer thereof, R¹, R², R³, and R⁴ are each independently —CH₃ or —CH₂CH₃ substituted with one R^{a}.

In some embodiments of a compound of Formula (II), or a pharmaceutically acceptable salt, solvate, or stereoisomer thereof, R¹, R², R³, and R⁴ are each independently —CH₃ or —CH₂CH₃ substituted with two R^{a}.

In some embodiments of a compound of Formula (II), or a pharmaceutically acceptable salt, solvate, or stereoisomer thereof, R¹, R², and R³ are each independently —CH₃ substituted with one R^{a}.

In some embodiments of a compound of Formula (II), or a pharmaceutically acceptable salt, solvate, or stereoisomer thereof, R⁴ is —CH₂CH₃ substituted with two R^{a}.

In some embodiments of a compound of Formula (II), or a pharmaceutically acceptable salt, solvate, or stereoisomer thereof, R¹ is alkyl substituted with a heteroaryl optionally substituted with one of more R^{b}.

In some embodiments of a compound of Formula (II), or a pharmaceutically acceptable salt, solvate, or stereoisomer thereof, R¹ is alkyl substituted with an indolyl optionally substituted with one of more R^{b}.

In some embodiments of a compound of Formula (II), or a pharmaceutically acceptable salt, solvate, or stereoisomer thereof, R¹ is alkyl substituted with an indolyl optionally substituted with one R^{b}.

In some embodiments of a compound of Formula (II), or a pharmaceutically acceptable salt, solvate, or stereoisomer thereof, R² is alkyl substituted with an aryl optionally substituted with one of more R^{b}.

In some embodiments of a compound of Formula (II), or a pharmaceutically acceptable salt, solvate, or stereoisomer thereof, R² is alkyl substituted with a phenyl optionally substituted with one R^{b}.

In some embodiments of a compound of Formula (II), or a pharmaceutically acceptable salt, solvate, or stereoisomer thereof, R² is alkyl substituted with a phenyl optionally substituted with one of more R^{b}.

In some embodiments of a compound of Formula (II), or a pharmaceutically acceptable salt, solvate, or stereoisomer thereof, R³ is alkyl substituted with a heteroaryl optionally substituted with one of more R^{b}.

In some embodiments of a compound of Formula (II), or a pharmaceutically acceptable salt, solvate, or stereoisomer thereof, R³ is alkyl substituted with a pyridyl optionally substituted with one of more R^{b}.

In some embodiments of a compound of Formula (II), or a pharmaceutically acceptable salt, solvate, or stereoisomer thereof, R³ is alkyl substituted with a pyridyl optionally substituted with one R^{b}.

In some embodiments of a compound of Formula (II), or a pharmaceutically acceptable salt, solvate, or stereoisomer thereof, R⁴ is alkyl substituted with a heteroaryl optionally substituted with one of more R^{b}.

In some embodiments of a compound of Formula (II), or a pharmaceutically acceptable salt, solvate, or stereoisomer thereof, R⁴ is alkyl substituted with an indolyl optionally substituted with one of more R^{b}.

In some embodiments of a compound of Formula (II), or a pharmaceutically acceptable salt, solvate, or stereoisomer thereof, R⁴ is alkyl substituted with R⁶ and with a heteroaryl optionally substituted with one of more R^{b}.

In some embodiments of a compound of Formula (II), or a pharmaceutically acceptable salt, solvate, or stereoisomer thereof, R⁴ is alkyl substituted with R⁶ and with an indolyl optionally substituted with one of more R^{b}.

In some embodiments of a compound of Formula (II), or a pharmaceutically acceptable salt, solvate, or stereoisomer thereof, R⁴ is alkyl substituted with R⁶ and with an indolyl optionally substituted with one R^{b}.

In some embodiments of a compound of Formula (II), or a pharmaceutically acceptable salt, solvate, or stereoisomer thereof, R^{b} is —H, —OH, —OCH₃, —OC₂H₅, —F, —Cl, —Br, —I, —NH₂, —S(═O)₂NH₂, —CF₃, —C(═O)NH₂, —NHC(═O)H, —NHC(═O)CH₃, —NO₂, —S(═O)CH₃, —NHS(═O)₂CH₃, —OCF₃, —OCH(CH₃)₂, —CH(CH₃)₂, —CH₂CH₂CH₃, —OCH₂CH₂CH₃, —OCH₂OCH₃, or —OCH₂CH₂OCH₃.

In some embodiments of a compound of Formula (II), or a pharmaceutically acceptable salt, solvate, or stereoisomer thereof, R^{b} is —H, —OH, —OCH₃, —F, or —Cl.

In some embodiments of a compound of Formula (II), or a pharmaceutically acceptable salt, solvate, or stereoisomer thereof, R⁶ is —H, —OH, —OCH₃, —OC₂H₅, —F, —Cl, —Br, —I, —NH₂, S(═O)₂NH₂, —CF₃, —C(═O)NH₂, —NHC(═O)H, —NHC(═O)CH₃, —NO₂, —S(═O)CH₃, —NHS(═O)₂CH₃, —OCF₃, —OCH(CH₃)₂, —CH(CH₃)₂, —CH₂CH₂CH₃, —OCH₂CH₂CH₃, —OCH₂OCH₃, —OCH₂CH₂OCH₃, —NHC(═O)H, or —NHC(═O)CH₃.

In some embodiments of a compound of Formula (II), or a pharmaceutically acceptable salt, solvate, or stereoisomer thereof, R⁶ is —H or —C(═O)NH₂.

In some embodiments of a compound of Formula (II), or a pharmaceutically acceptable salt, solvate, or stereoisomer thereof, X¹, X², X³, and X⁴ are each independently —C(═O)NH— or —NHC(═O)—.

In some embodiments of a compound of Formula (II), or a pharmaceutically acceptable salt, solvate, or stereoisomer thereof, X¹, X², X³, and X⁴ are each —C(═O)NH—.

In some embodiments of a compound of Formula (II), or a pharmaceutically acceptable salt, solvate, or stereoisomer thereof, R⁵ is —H. In some embodiments of a compound of Formula (II), or a pharmaceutically acceptable salt, solvate, or stereoisomer thereof, R⁵ is —C(═O)(CH₂CH₂O)ₖCH₃. In some embodiments of a compound of Formula (II), or a pharmaceutically acceptable salt, solvate, or stereoisomer thereof, k is 1 to 100. In some embodiments of a compound of Formula (II), or a pharmaceutically acceptable salt, solvate, or stereoisomer thereof, k is 100 to 500. In some embodiments of a compound of Formula (II), or a pharmaceutically acceptable salt, solvate, or stereoisomer thereof, k is 500 to 1000.

In some embodiments of a compound of Formula (II), or a pharmaceutically acceptable salt, solvate, or stereoisomer thereof, the compound is represented by the following formula:

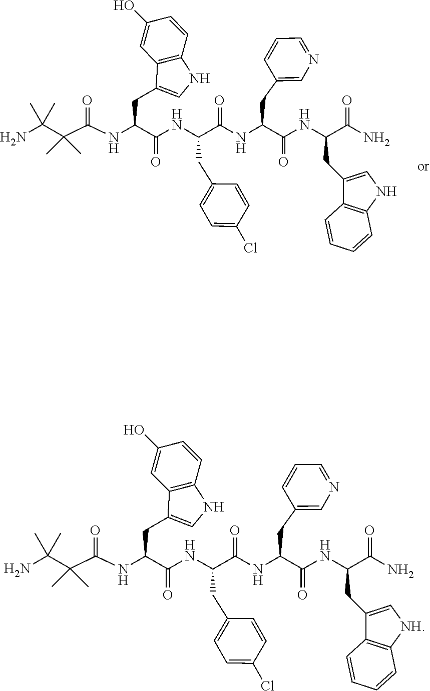 [SMILES: CC(C)(N)C(C)(C)C(=O)N[C@@H](Cc1c[nH]c2ccc(O)cc12)C(=O)N[C@@H](Cc1ccc(Cl)cc1)C(=O)N[C@@H](Cc1cccnc1)C(=O)N[C@H](Cc1c[nH]c2ccccc12)C(N)=O ; CC(C)(N)C(C)(C)C(=O)N[C@@H](Cc1c[nH]c2ccc(O)cc12)C(=O)N[C@@H](Cc1ccc(Cl)cc1)C(=O)N[C@@H](Cc1cccnc1)C(=O)N[C@H](Cc1c[nH]c2ccccc12)C(N)=O]

In another aspect, provided herein is a compound of formula (III), or a pharmaceutically acceptable salt, solvate, or stereoisomer thereof:

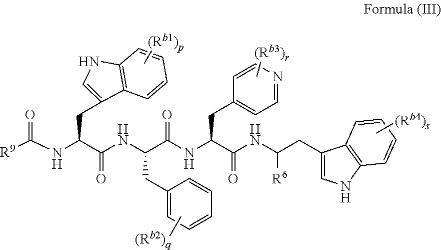

wherein
R⁶ is —H, —OH, —OCH₃, —OC₂H₅, —F, —Cl, —Br, —I, —NH₂, S(═O)₂NH₂, —CF₃, —C(═O)NH₂, —NHC(═O)H, —NHC(═O)CH₃, —NO₂, —S(═O)CH₃, —NHS(═O)₂CH₃, —OCF₃, —OCH(CH₃)₂, —CH(CH₃)₂, —CH₂CH₂CH₃, —OCH₂CH₂CH₃, —OCH₂OCH₃, —OCH₂CH₂OCH₃, —NHC(═O)H, or —NHC(═O)CH₃;
R⁹ is a natural or non-natural amino acid or a di-peptide;
R^{b1}, R^{b2}, R^{b3}, and R^{b4} are independently —H, —OH, —OCH₃, —OC₂H₅, —F, —Cl, —Br, —I, —NH₂, —S(═O)₂NH₂, —CF₃, —C(═O)NH₂, —NHC(═O)H, —NHC(═O)CH₃, —NO₂, —S(═O)CH₃, —NHS(═O)₂CH₃, —OCF₃, —OCH(CH₃)₂, —CH(CH₃)₂, —CH₂CH₂CH₃, —OCH₂CH₂CH₃, —OCH₂OCH₃, or —OCH₂CH₂OCH₃; and
p, q, r, and s are each independently 1-3.

In some embodiments of a compound of Formula (III), R⁹ comprises a glycine amino acid residue.

In some embodiments of a compound of Formula (III), R⁹ comprises a substituted glycine amino acid residue.

In some embodiments of a compound of Formula (III), R⁹ comprises two glycine amino acid residues.

In some embodiments of a compound of Formula (III), R⁹ comprises an alanine amino acid residue.

In some embodiments of a compound of Formula (III), R⁹ comprises an aminobutyric acid.

In some embodiments of a compound of Formula (III), or a pharmaceutically acceptable salt, solvate, or stereoisomer thereof, R⁶ is —H or —C(═O)NH₂.

In some embodiments of a compound of Formula (III), or a pharmaceutically acceptable salt, solvate, or stereoisomer thereof, R^{b1}, R^{b2}, R^{b3}, and R^{b4} are independently —H, —OH, —OCH₃, —F, or —Cl.

In some embodiments of a compound of Formula (III), or a pharmaceutically acceptable salt, solvate, or stereoisomer thereof, p is 1; and R^{b1} is —OH.

In some embodiments of a compound of Formula (III), or a pharmaceutically acceptable salt, solvate, or stereoisomer thereof, q is 1; and R^{b2} —Cl.

In some embodiments of a compound of Formula (III), or a pharmaceutically acceptable salt, solvate, or stereoisomer thereof, r is 1; and R^{b3} is —H.

In some embodiments of a compound of Formula (III), or a pharmaceutically acceptable salt, solvate, or stereoisomer thereof, s is 1; and R^{b4} is —H, —OH, —OCH₃, —F, or —Cl.

Further embodiments provided herein include combinations of one or more of the particular embodiments set forth above.

In some embodiments, the compound disclosed herein has the structure provided in

TABLE 1

| Comp. | Name | Structure |
|---|---|---|
| 1 | 3-amino-N-((S)-1-((S)-1- ((S)-1-((R)-1-amino-3- (1H-indol-3-yl)-1- oxopropan-2-ylamino)-1- oxo-3-(pyridin-3- yl)propan-2-ylamino)-3- (4-chlorophenyl)-1- oxopropan-2-ylamino)-3- (5-hydroxy-1H-indol-3- yl)-1-oxopropan-2-yl)- 2,2,3- trimethylbutanamide | 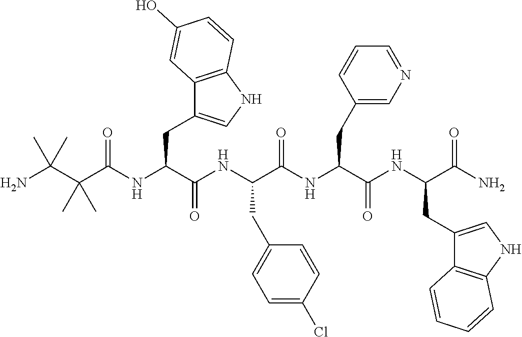 [SMILES: CC(C)(N)C(C)(C)C(=O)N[C@@H](Cc1c[nH]c2ccc(O)cc12)C(=O)N[C@@H](Cc1ccc(Cl)cc1)C(=O)N[C@@H](Cc1cccnc1)C(=O)N[C@H](Cc1c[nH]c2ccccc12)C(N)=O] |
| 2 | 3-amino-N-((S)-1-((S)-1- ((S)-1-((R)-1-amino-3- (1H-indol-3-yl)-1- oxopropan-2-ylamino)-1- oxo-3-(pyridin-2- yl)propan-2-ylamino)-3- (4-chlorophenyl)-1- oxopropan-2-ylamino)-3- (5-hydroxy-1H-indol-3- yl)-1-oxopropan-2-yl)- 2,2,3- trimethylbutanamide | 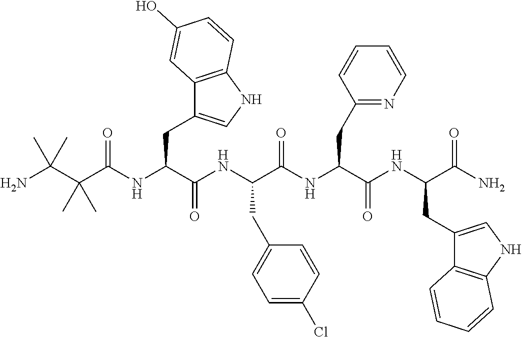 [SMILES: CC(C)(N)C(C)(C)C(=O)N[C@@H](Cc1c[nH]c2ccc(O)cc12)C(=O)N[C@@H](Cc1ccc(Cl)cc1)C(=O)N[C@@H](Cc1ccccn1)C(=O)N[C@H](Cc1c[nH]c2ccccc12)C(N)=O] |
| 3 | 4-amino-N-((S)-1-((S)-1- ((S)-1-((R)-1-amino-3- (1H-indol-3-yl)-1- oxopropan-2-ylamino)-1- oxo-3-(pyridin-4- yl)propan-2-ylamino)-3- (4-chlorophenyl)-1- oxopropan-2-ylamino)-3- (5-hydroxy-1H-indol-3- yl)-1-oxopropan-2- yl)butanamide | 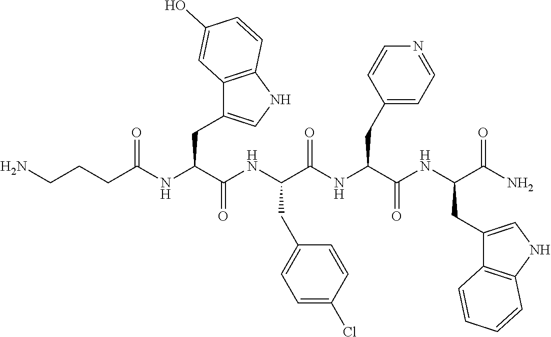 [SMILES: NCCCC(=O)N[C@@H](Cc1c[nH]c2ccc(O)cc12)C(=O)N[C@@H](Cc1ccc(Cl)cc1)C(=O)N[C@@H](Cc1ccncc1)C(=O)N[C@H](Cc1c[nH]c2ccccc12)C(N)=O] |
| 4 | 5-amino-N-((S)-1-((S)-1- ((S)-1-((R)-1-amino-3- (1H-indol-3-yl)-1- oxopropan-2-ylamino)-1- oxo-3-(pyridin-4- yl)propan-2-ylamino)-3- (4-chlorophenyl)-1- oxopropan-2-ylamino)-3- (5-hydroxy-1H-indol-3- yl)-1-oxopropan-2- yl)pentanamide | 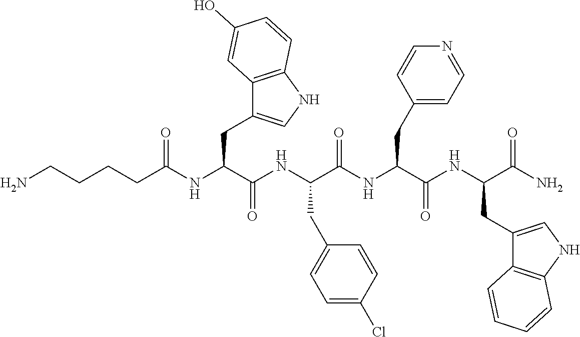 [SMILES: NCCCCC(=O)N[C@@H](Cc1c[nH]c2ccc(O)cc12)C(=O)N[C@@H](Cc1ccc(Cl)cc1)C(=O)N[C@@H](Cc1ccncc1)C(=O)N[C@H](Cc1c[nH]c2ccccc12)C(N)=O] |
| 5 | N-((S)-1-((S)-1-((S)-1-(2- (1H-indol-3- yl)ethylamino)-1-oxo-3- (pyridin-4-yl)propan-2- ylamino)-3-(4- chlorophenyl)-1- oxopropan-2-ylamino)-3- (5-hydroxy-1H-indol-3- yl)-1-oxopropan-2-yl)-4- aminobutanamide | 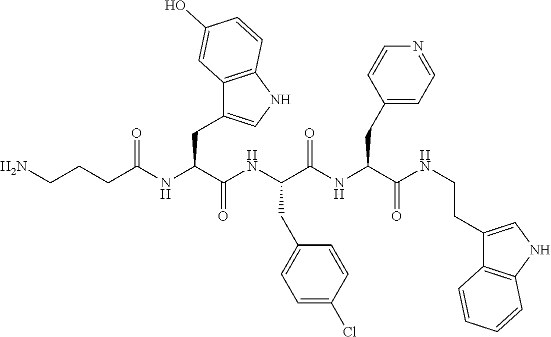 [SMILES: NCCCC(=O)N[C@@H](Cc1c[nH]c2ccc(O)cc12)C(=O)N[C@@H](Cc1ccc(Cl)cc1)C(=O)N[C@@H](Cc1ccncc1)C(=O)NCCc1c[nH]c2ccccc12] |
| 6 | 4-amino-N-((S)-1-((S)-3- (4-chlorophenyl)-1-((S)- 1-(2-(5-fluoro-1H-indol- 3-yl)ethylamino)-1-oxo-3- (pyridin-4-yl)propan-2- ylamino)-1-oxopropan-2- ylamino)-3-(5-hydroxy- 1H-indol-3-yl)-1- oxopropan-2- yl)butanamide | 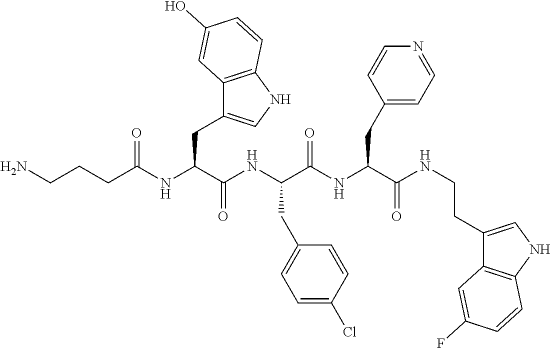 [SMILES: NCCCC(=O)N[C@@H](Cc1c[nH]c2ccc(O)cc12)C(=O)N[C@@H](Cc1ccc(Cl)cc1)C(=O)N[C@@H](Cc1ccncc1)C(=O)NCCc1c[nH]c2ccc(F)cc12] |
| 7 | 4-amino-N-((S)-1-((S)-3- (4-chlorophenyl)-1-((S)- 1-(2-(5-methoxy-1H- indol-3-yl)ethylamino)-1- oxo-3-(pyridin-4- yl)propan-2-ylamino)-1- oxopropan-2-ylamino)-3- (5-hydroxy-1H-indol-3- yl)-1-oxopropan-2- yl)butanamide | 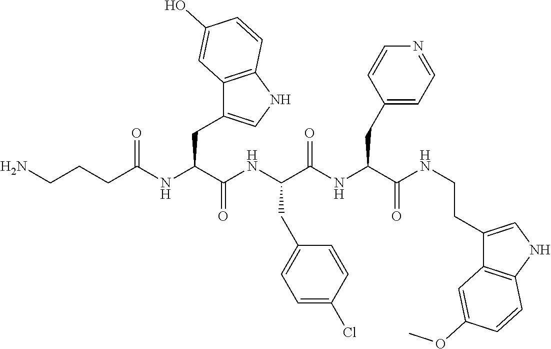 [SMILES: COc1ccc2[nH]cc(CCNC(=O)[C@H](Cc3ccncc3)NC(=O)[C@H](Cc3ccc(Cl)cc3)NC(=O)[C@H](Cc3c[nH]c4ccc(O)cc34)NC(=O)CCCN)c2c1] |
| 8 | 4-amino-N-((S)-1-((S)-3- (4-chlorophenyl)-1-((S)- 1-(2-(5-hydroxy-1H- indol-3-yl)ethylamino)-1- oxo-3-(pyridin-4- yl)propan-2-ylamino)-1- oxopropan-2-ylamino)-3- (5-hydroxy-1H-indol-3- yl)-1-oxopropan-2- yl)butanamide | 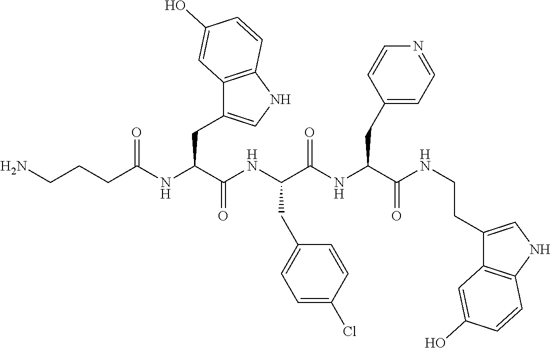 [SMILES: NCCCC(=O)N[C@@H](Cc1c[nH]c2ccc(O)cc12)C(=O)N[C@@H](Cc1ccc(Cl)cc1)C(=O)N[C@@H](Cc1ccncc1)C(=O)NCCc1c[nH]c2ccc(O)cc12] |
| 9 | 4-amino-N-((S)-1-((S)-1- ((S)-1-(2-(5-chloro-1H- indol-3-yl)ethylamino)-1- oxo-3-(pyridin-4- yl)propan-2-ylamino)-3- (4-chlorophenyl)-1- oxopropan-2-ylamino)-3- (5-hydroxy-1H-indol-3- yl)-1-oxopropan-2- yl)butanamide | 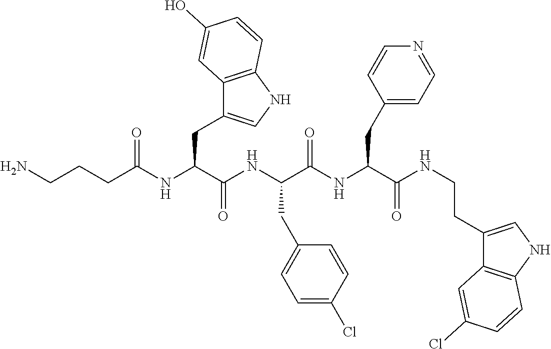 [SMILES: NCCCC(=O)N[C@@H](Cc1c[nH]c2ccc(O)cc12)C(=O)N[C@@H](Cc1ccc(Cl)cc1)C(=O)N[C@@H](Cc1ccncc1)C(=O)NCCc1c[nH]c2ccc(Cl)cc12] |
| 10 | 4-amino-N-((S)-1-((S)-3- (4-chlorophenyl)-1-((S)- 1-(2-(6-methoxy-1H- indol-3-yl)ethylamino)-1- oxo-3-(pyridin-4- yl)propan-2-ylamino)-1- oxopropan-2-ylamino)-3- (5-hydroxy-1H-indol-3- yl)-1-oxopropan-2- yl)butanamide | 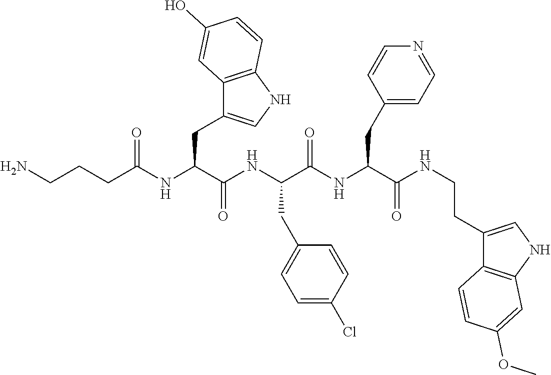 [SMILES: COc1ccc2c(CCNC(=O)[C@H](Cc3ccncc3)NC(=O)[C@H](Cc3ccc(Cl)cc3)NC(=O)[C@H](Cc3c[nH]c4ccc(O)cc34)NC(=O)CCCN)c[nH]c2c1] |
| 11 | 4-amino-N-((S)-1-((S)-3- (4-chlorophenyl)-1-((S)- 1-(2-(naphthalen-1- yl)ethylamino)-1-oxo-3- (pyridin-4-yl)propan-2- ylamino)-1-oxopropan-2- ylamino)-3-(5-hydroxy- 1H-indol-3-yl)-1- oxopropan-2- yl)butanamide | 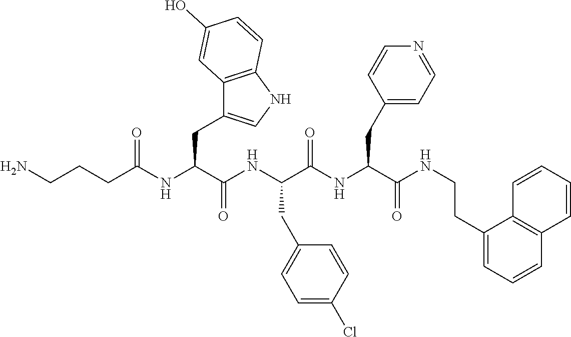 [SMILES: NCCCC(=O)N[C@@H](Cc1c[nH]c2ccc(O)cc12)C(=O)N[C@@H](Cc1ccc(Cl)cc1)C(=O)N[C@@H](Cc1ccncc1)C(=O)NCCc1cccc2ccccc12] |

### PEGylated Compounds

In some embodiments, compounds described herein are conjugated with polyethylene glycol (PEG) to change their pharmacokinetic and pharmacodynamic profiles. In some embodiments, the PEGylated compounds described herein have improved pharmacokinetic and pharmacodynamic profiles by increasing water solubility, protecting from enzymatic degradation, reducing renal clearance and limiting immunogenic and antigenic reactions. In some embodiments, the PEGylated compounds described herein show increased half-life, decreased plasma clearance, and different biodistribution, in comparison with non-PEGylated counterparts. In some embodiments, the PEGylated compounds have the following formula:

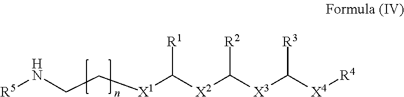

wherein
X¹, X², X³, and X⁴ are each independently —C(═O)NH—, —NHC(═O)—, —S(═O)₂NH—, —O—, —CH₂—, —CH₂CH₂—, —OCH₂—, —CH₂O—, —S—, —S(═O)₂—, or —C(═O)NHS(═O)₂—;
R¹, R², R³, and R⁴ are each independently alkyl, heterocycloalkyl, or cycloalkyl; wherein the alkyl, heterocycloalkyl, and cycloalkyl are optionally substituted with one of more R^{a};
R⁵ comprises polyethylene glycol (PEG);
R⁶ is —H, halogen, alkyl, haloalkyl, alkoxy, haloalkoxy, —OH, —NH₂, S(═O)₂NH₂, —C(═O)NH₂, —NHC(═O)H, —NHC(═O)CH₃, —NO₂, —S(═O)CH₃, —NHS(═O)₂CH₃, —OCH₂OCH₃, —OCH₂CH₂OCH₃, —NHC(═O)H, or —NHC(═O)CH₃;
R^{a} is R⁶, aryl or heteroaryl; wherein the aryl or heteroaryl are optionally substituted with one or more R^{b};
R^{b} is —H, halogen, alkyl, haloalkyl, alkoxy, haloalkoxy, —OH, —NH₂, —S(═O)₂NH₂, —C(═O)NH₂, —NHC(═O)H, —NHC(═O)CH₃, —NO₂, —S(═O)CH₃, —NHS(═O)₂CH₃, —OCH₂OCH₃, or —OCH₂CH₂OCH₃; and
n is 0-4.

In some embodiments of a compound of Formula (IV), or a pharmaceutically acceptable salt, solvate, or stereoisomer thereof, R⁵ is —C(═O)(CH₂CH₂O)ₖCH₃; and k is 1-1000. In some embodiments of a compound of Formula (IV), or a pharmaceutically acceptable salt, solvate, or stereoisomer thereof, k is 1 to 100. In some embodiments of a compound of Formula (IV), or a pharmaceutically acceptable salt, solvate, or stereoisomer thereof, k is 100 to 500. In some embodiments of a compound of Formula (IV), or a pharmaceutically acceptable salt, solvate, or stereoisomer thereof, k is 500 to 1000. In some embodiments of a compound of Formula (IV), or a pharmaceutically acceptable salt, solvate, or stereoisomer thereof, k is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100. In some embodiments of a compound of Formula (IV), or a pharmaceutically acceptable salt, solvate, or stereoisomer thereof, k is at least 10, at least 20, at least 30, at least 40, at least 50, at least 60, at least 70, at least 80, at least 90, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, or at least 1000.

### Preparation of the Compounds

The compounds used in the reactions described herein are made according to known organic synthesis techniques, starting from commercially available chemicals and/or from compounds described in the chemical literature. “Commercially available chemicals” are obtained from standard commercial sources including Acros Organics (Geel, Belgium), Aldrich Chemical (Milwaukee, Wis., including Sigma Chemical and Fluka), Apin Chemicals Ltd. (Milton Park, UK), Ark Pharm, Inc. (Libertyville, Ill.), Avocado Research (Lancashire, U.K.), BDH Inc. (Toronto, Canada), Bionet (Cornwall, U.K.), Chemservice Inc. (West Chester, Pa.), Combi-blocks (San Diego, Calif.), Crescent Chemical Co. (Hauppauge, N.Y.), eMolecules (San Diego, Calif.), Fisher Scientific Co. (Pittsburgh, Pa.), Fisons Chemicals (Leicestershire, UK), Frontier Scientific (Logan, Utah), ICN Biomedicals, Inc. (Costa Mesa, Calif.), Key Organics (Cornwall, U.K.), Lancaster Synthesis (Windham, N.H.), Matrix Scientific, (Columbia, S.C.), Maybridge Chemical Co. Ltd. (Cornwall, U.K.), Parish Chemical Co. (Orem, Utah), Pfaltz & Bauer, Inc. (Waterbury, Conn.), Polyorganix (Houston, Tex.), Pierce Chemical Co. (Rockford, Ill.), Riedel de Haen AG (Hanover, Germany), Ryan Scientific, Inc. (Mount Pleasant, S.C.), Spectrum Chemicals (Gardena, Calif.), Sundia Meditech, (Shanghai, China), TCI America (Portland, Oreg.), Trans World Chemicals, Inc. (Rockville, Md.), and WuXi (Shanghai, China).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are also identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Further Forms of Compounds Disclosed Herein

### Isomers

Furthermore, in some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers. The compounds described herein include all possible tautomers within the formulas described herein.

In some situations, the compounds described herein possess one or more chiral centers and each center exists in the R configuration, or S configuration. In some embodiments, the compounds described herein possess three chiral centers and each center exists in the R configuration, or S configuration. In some embodiments, the compounds described herein possess four chiral centers and each center exists in the R configuration, or S configuration. In some embodiments, the compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that are incorporated into compounds of the invention include isotopes of hydrogen, carbon, nitrogen, oxygen, phosphorous, sulfur, fluorine and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and pharmaceutically acceptable salts, esters, solvate, hydrates or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i. e., ³H and carbon-14, i. e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compounds, pharmaceutically acceptable salt, ester, solvate, hydrate or derivative thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds of the invention, or by separately reacting a purified compound in its free form with a suitable acid or base, and isolating the salt thus formed.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran or methanol. In addition, the compounds provided herein exist in unsolvated as well as solvated forms. In general, the solvated forms are considered equivalent to the unsolvated forms for the purposes of the compounds and methods provided herein.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, twentyfirst Ed (Lippincott Williams & Wilkins 2012); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be effected by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, compounds described herein can be administered locally to the area in need of treatment, by for example, local infusion during surgery, topical application such as creams or ointments, injection, catheter, or implant. The administration can also be by direct injection at the site of a diseased tissue or organ.

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

Pharmaceutical compositions may also be formulated in rectal compositions such as suppositories or retention enemas, e.g., containing conventional suppository bases such as cocoa butter, polyethylene glycol, or other glycerides.

Pharmaceutical compositions may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Pharmaceutical compositions suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation.

Pharmaceutical compositions for administration by inhalation are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, pharmaceutical preparations may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

### Nanoparticles

In some embodiments, the pharmaceutical compositions described herein are in the form of nanoparticles. Nanoparticles are submicroscopic solid particles with size ranging from 10 nm to 1 μm. The size of the nanoparticles allows for intravenous administration with little risk of embolism. Materials used in the preparation of nanoparticles are sterilizable, nontoxic, and biodegradable, such as, albumin, ethylcellulose, casein, gelatin, polyesters, polyanhydrides, and polyalkyl cyanoacrylates. In some embodiments, encapsulation into a nanoparticle protects the compounds described herein against enzymatic degradation and achieves controlled release. In some embodiments, the nanoparticles are nanocapsules or nanospheres. Nanocapsules are vesicular systems in which the compounds described herein are surrounded by a membrane. Nanospheres are matrix systems in which the compounds described herein are dispersed throughout the particles. There are a number of methods for preparing nanoparticles such as solvent evaporation, organic phase separation, interfacial polymerization, emulsion polymerization, and spray drying. In some embodiments, PLGA-based copolymers are used as the biodegradable or bioerodible polymers in the nanoparticles. In some embodiments, PLGA-based nanoparticles are useful for controlled delivery of the compounds described herein. In some embodiments, the compounds described herein are released from the nanoparticles via polymer erosion or degradation, self diffusion through pores, or released from the polymer surface. In some embodiments, an initial burst is observed because of solubilization of free drug near the surface of the nanoparticle that is followed by disintegration of the matrix. In some embodiments, the release profile is biphasic with an initial high release rate followed by a period of significant release due to polymer degradation.

In some embodiments, the nanoparticles comprise compounds described herein conjugated with polyethylene glycol (PEG). In some embodiments, compounds described herein are conjugated with polyethylene glycol (PEG) to change their pharmacokinetic and pharmacodynamic profiles. In some embodiments, the PEGylated compounds described herein have improved pharmacokinetic and pharmacodynamic profiles by increasing water solubility, protecting from enzymatic degradation, reducing renal clearance and limiting immunogenic and antigenic reactions. In some embodiments, the PEGylated compounds described herein show increased half-life, decreased plasma clearance, and different biodistribution, in comparison with non-PEGylated counterparts. In some embodiments, the PEGylated compounds comprised in the nanoparticles have the following formula:

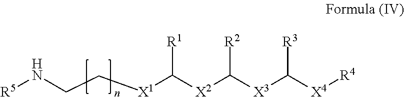

wherein
X¹, X², X³, and X⁴ are each independently —C(═O)NH—, —NHC(═O)—, —S(═O)₂NH—, —O—, —CH₂—, —CH₂CH₂—, —OCH₂—, —CH₂O—, —S—, —S(═O)₂—, or —C(═O)NHS(═O)₂—;
R¹, R², R³, and R⁴ are each independently alkyl, heterocycloalkyl, or cycloalkyl; wherein the alkyl, heterocycloalkyl, and cycloalkyl are optionally substituted with one of more R^{a};
R⁵ comprises polyethylene glycol chains (PEG);
R⁶ is —H, halogen, alkyl, haloalkyl, alkoxy, haloalkoxy, —OH, —NH₂, S(═O)₂NH₂, —C(═O)NH₂, —NHC(═O)H, —NHC(═O)CH₃, —NO₂, —S(═O)CH₃, —NHS(═O)₂CH₃, —OCH₂OCH₃, —OCH₂CH₂OCH₃, —NHC(═O)H, or —NHC(═O)CH₃;
R^{a} is R⁶, aryl or heteroaryl; wherein the aryl or heteroaryl are optionally substituted with one or more R^{b};
R^{b} is —H, halogen, alkyl, haloalkyl, alkoxy, haloalkoxy, —OH, —NH₂, —S(═O)₂NH₂, —C(═O)NH₂, —NHC(═O)H, —NHC(═O)CH₃, —NO₂, —S(═O)CH₃, —NHS(═O)₂CH₃, —OCH₂OCH₃, or —OCH₂CH₂OCH₃; and
n is 0-4.

In some embodiments of a compound of Formula (IV), or a pharmaceutically acceptable salt, solvate, or stereoisomer thereof, R⁵ is —C(═O)(CH₂CH₂O)ₖCH₃; and k is 1-1000. In some embodiments of a compound of Formula (IV), or a pharmaceutically acceptable salt, solvate, or stereoisomer thereof, k is 1 to 100. In some embodiments of a compound of Formula (IV), or a pharmaceutically acceptable salt, solvate, or stereoisomer thereof, k is 100 to 500. In some embodiments of a compound of Formula (IV), or a pharmaceutically acceptable salt, solvate, or stereoisomer thereof, k is 500 to 1000. In some embodiments of a compound of Formula (IV), or a pharmaceutically acceptable salt, solvate, or stereoisomer thereof, k is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100. In some embodiments of a compound of Formula (IV), or a pharmaceutically acceptable salt, solvate, or stereoisomer thereof, k is at least 10, at least 20, at least 30, at least 40, at least 50, at least 60, at least 70, at least 80, at least 90, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, or at least 1000.

It should be understood that in addition to the ingredients particularly mentioned above, the compounds and compositions described herein may include other agents conventional in the art having regard to the type of formulation in question, for example those suitable for oral administration may include flavoring agents.

### Methods of Treatment and Treatment Regiments

In some embodiments, described herein are methods for the treatment of diseases or conditions where the EphA4 receptor is involved including but not limited to abnormal blood clotting, cancer, spinal cord injury, ALS, Alzheimer's disease, or traumatic brain injury.

In some embodiments, described herein are methods for the treatment of ALS, comprising administering a therapeutically effective amount of a compound described herein, or a pharmaceutically acceptable salt, solvate, or stereoisomer thereof to a subject in need thereof.

In some embodiments, described herein are methods for the treatment of Alzheimer's disease, comprising administering a therapeutically effective amount of a compound described herein, or a pharmaceutically acceptable salt, solvate, or stereoisomer thereof to a subject in need thereof.

In some embodiments, described herein are methods for the treatment of abnormal blood clotting, comprising administering a therapeutically effective amount of a compound described herein, or a pharmaceutically acceptable salt, solvate, or stereoisomer thereof to a subject in need thereof.

In some embodiments, described herein are methods for the treatment of cancer, comprising administering a therapeutically effective amount of a compound described herein, or a pharmaceutically acceptable salt, solvate, or stereoisomer thereof to a subject in need thereof. In some embodiments, the cancer is gastric cancer, breast cancer, pancreatic cancer, or prostate cancer.

In some embodiments, described herein are methods for the treatment of spinal cord injury, comprising administering a therapeutically effective amount of a compound described herein, or a pharmaceutically acceptable salt, solvate, or stereoisomer thereof to a subject in need thereof.

In some embodiments, described herein are methods for the treatment of traumatic brain injury, comprising administering a therapeutically effective amount of a compound described herein, or a pharmaceutically acceptable salt, solvate, or stereoisomer thereof to a subject in need thereof.

Methods for treating any of the diseases or conditions described herein in a mammal in need of such treatment, involves administration of pharmaceutical compositions that include at least one compound described herein or a pharmaceutically acceptable salt, solvate, or stereoisomer thereof, in therapeutically effective amounts to said mammal.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of the disease being treated and is currently in remission, a pharmaceutical composition comprising a compound described herein, or a pharmaceutically acceptable salt thereof, in order to prevent a return of the symptoms of the disease or condition.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

In certain embodiments wherein a patient's status does improve, the dose of drug being administered is temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering one of the compounds described herein with another agent (which also includes a therapeutic regimen) that also has therapeutic benefit.

In any case, regardless of the disease, disorder or condition being treated, the overall benefit experienced by the patient may be additive of the two therapeutic agents or the patient may experience a synergistic benefit.

It is understood that the dosage regimen to treat, prevent, or ameliorate the condition(s) for which relief is sought, is modified in accordance with a variety of factors (e.g. the disease, disorder or condition from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated and so forth. In additional embodiments, when co-administered with one or more other therapeutic agents, the compound provided herein is administered either simultaneously with the one or more other therapeutic agents, or sequentially.

The compounds described herein, or a pharmaceutically acceptable salt thereof, as well as combination therapies, are administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a compound described herein or a formulation containing the compound is administered for at least 2 weeks, about 1 month to about 5 years.

### EXAMPLES

The following examples are provided for illustrative purposes only and not to limit the scope of the claims provided herein.

### Example 1. Synthesis of 4-amino-N—((S)-1-(((S)-3-(4-chlorophenyl)-1-(((S)-1-((2-(5-methoxy-1H-indol-3-yl)ethyl)amino)-1-oxo-3-(pyridin-4-yl)propan-2-yl)amino)-1-oxopropan-2-yl)amino)-3-(5-hydroxy-1H-indol-3-yl)-1-oxopropan-2-yl)butanamide (Compound 7)

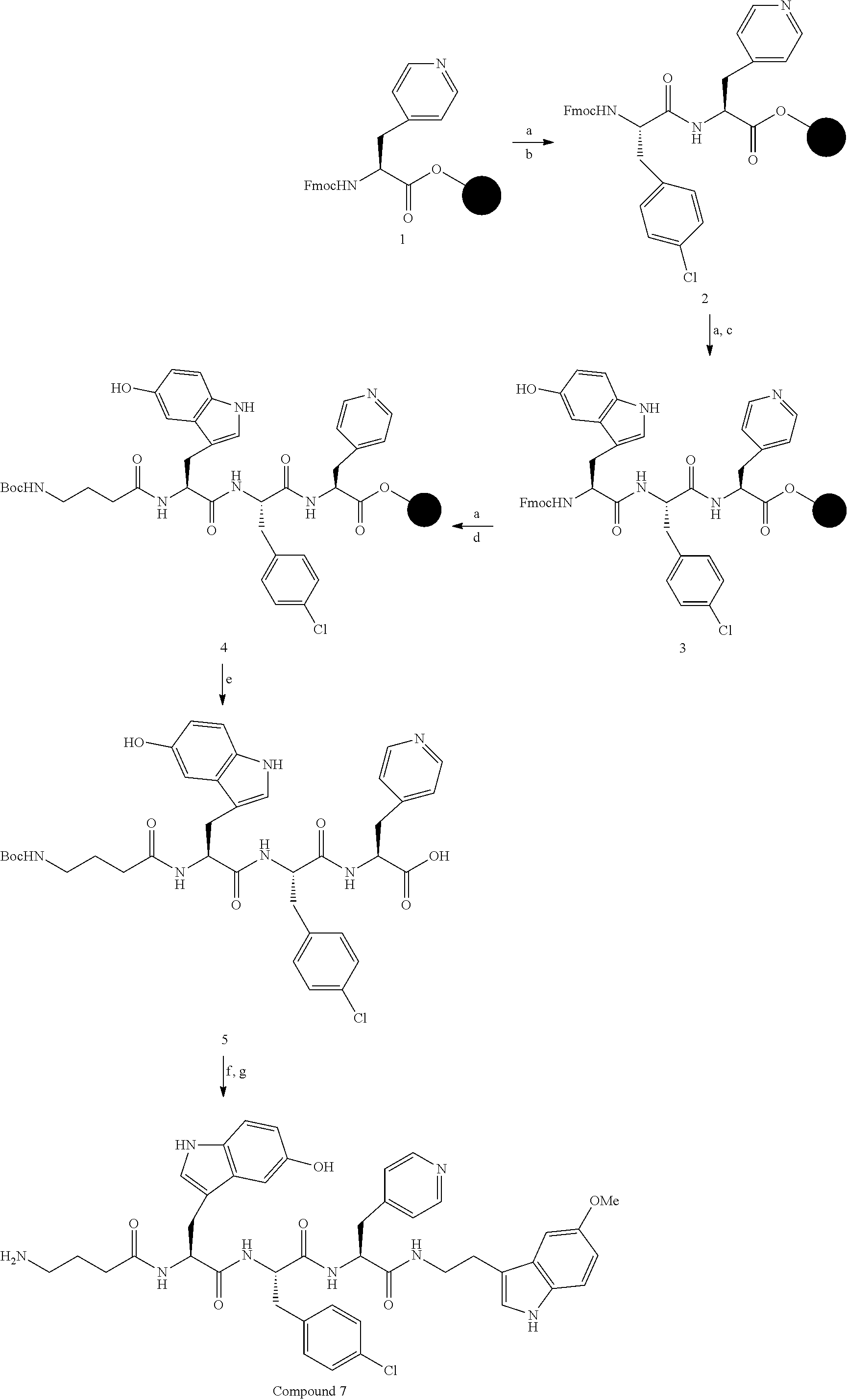

Reagents and conditions: (a) 20% piperidine in DMF; (b) Fmoc-L-4-chlorophenylalanine, Oxyma Pure, DIC, DIEA, DMF, rt, 2 h; (c) Fmoc-L-5-hydroxytryptophan, Oxyma pure, DIC, DIEA, DMF, rt, 2 h.; (d) Fmoc-gamma-Abu-OH, OXyma Pure, DIC, DIEA, DMF, rt, 2 h.; (e) Acetic acid, DCM, Trifluoroethanol, rt, 1 h.; (f) 5-methoxytryptamine, Oxyma pure, EDC, DIEA, DMF, 12 h, rt.; (g) 50% TFA in DCM, Phenol, TIPS, H₂O, rt, 3 h. A mixture of Intermediate 5 (735 mg, 1 mmol), 5-methoxytryptamine hydrochloride (226 mg, 1 mmol), EDC (229 mg 1.2 mmol), oxyma pure (170 mg, 1.2 mmol), DIEA (387 mg, 3 mmol) in DMF (5 mL) was stirred at room temperature for 16 h. All liquids were removed in vacuo and the crude was used for the next step. The crude was treated with 50% TFA in dichloromethane (total volume 10 mL) in the presence of phenol (2%), TIPS (2%), and water (2%) for 3 h. After that TFA and dichloromethane were removed under reduced pressure and the peptide was washed with diethyl ether (3×20 mL) and dried under high vacuum. The crude was dissolved in DMSO and purified by preparative reverse phase HPLC using acetonitrile-water system with 0.1% TFA. Compound 7 was characterized by NMR and MALDI mass. Purity was >95%. ¹H NMR (600 MHz, CD₃OD) δ 1.78-1.88 (m, 2H), 2.31 (t, J=6.6 Hz, 2H), 2.74-2.2.99 (m, 9H), 3.10-3.15 (m, 1H), 3.18-3.22 (m, 1H), 3.40-3.45 (m 1H), 3.55-3.60 (m, 1H), 4.45 (t, J=7.2 Hz, 4.58-4.66 (m, 2H), 6.66 (dd, J=8.4 and 2.4 Hz, 1H), 6.79 (dd, J=8.4 and 2.4 Hz, 1H), 6.94 (d, J=2.4 Hz, 1H), 7.04 (s, 1H), 7.07 (s, 2H), 7.10 (d, J=8.4 Hz, 2H), 7.13 (d, J=9 Hz, 1H), 7.21 (d, J=7.8 Hz, 2H), 7.24 (d, J=8.4 Hz, 1H), 7.52 (d, J=6 Hz, 2H), 8.43 (d, J=6.6 Hz, 2H); ¹³C NMR (150 MHz, CD₃OD) δ 22.68, 24.57, 27.35, 31.84, 36.20, 37.57, 38.77, 39.72, 52.74, 53.89, 54.41, 54.91, 100.05, 102.21, 108.62, 111.11, 111.16, 111.33, 111.38, 111.61, 123.04, 124.03, 127.51, 127.66, 128.06, 128.09, 130.51, 131.46, 131.93, 132.19, 135.40, 141.29, 149.93, 153.58, 157.68, 169.58, 171.37, 172.41, 172.75; MALDI (m/z): 807 (M⁺).

Compounds 1, 2, 3, 4, 5, 6, 8, 9, 10, and 11 were synthesized as described for Example 1 using the appropriate starting materials.

### Example I. Binding and Displacement Data by FPA and ITC Measurements

Fluorescence Polarization Assay (FPA):

The EphA4 binding KYL peptide (KYLPYWPVLSSL, Murai et al. Mol. Cell. Neurosci. 2003, 24:1000-1011) was labeled at the N-terminus with fluorescein isothiocyanate (FITC) and purified by HPLC. For competitive binding assays, 1 μL of 200 μM EphA4 LBD was pre-incubated with the tested compounds at various concentrations in 98 μL PBS (pH=7.2) in 96-well black plates at room temperature for 10 min, and then 1 μL of 500 μM FITC labeled EphA4 peptide was added to produce a final volume of 100 μL. The KYL and DMSO were incubated in each assay as positive and negative controls, respectively. After 30 min of incubation at room temperature, the polarization values in millipolarization units were measured at excitation/emission wavelengths of 480/535 nm with a multilabel plate reader (PerkinElmer, Waltham, Mass., USA). Kᵢ value was determined by fitting the experimental data to a Sigmoidal dose-response (variable slope) nonlinear regression model (GraphPad Prism version 5.01 for Windows, GraphPad Software, San Diego, Calif., USA).

Isothermal Titration Calorimetry (ITC):

isothermal titration calorimetry (ITC) to measure binding constant was performed on a Model ITC200 calorimeter from Microcal/GE Life Sciences. When indicated, measurements were performed in a reverse way—i.e., the protein was titrated into the compound solution. A total of 8 μl EphA4 solution (1.65 mM) was injected into the cell containing 165 M of compound per injection. All titrations were performed at 25° C. in PBS buffer supplemented with 10% DMSO. Experimental data were analyzed using Microcal Origin software provided by the ITC manufacturer (Microcal).

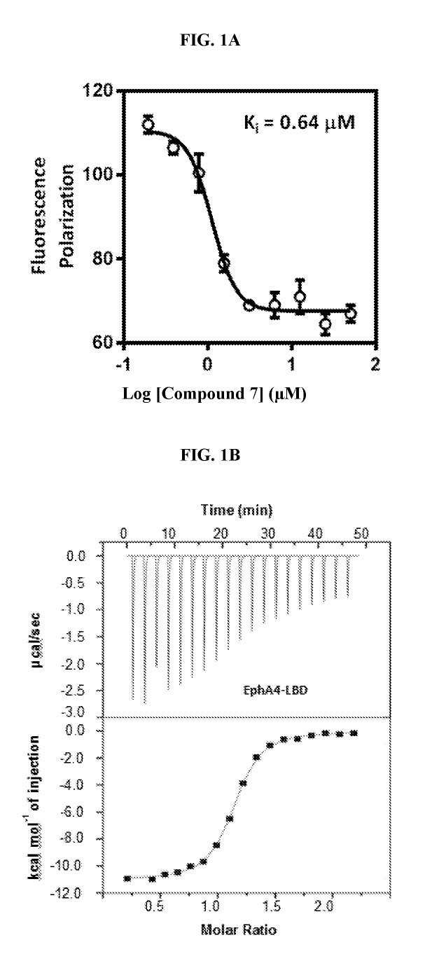

FIG. 1: Binding studies with Compound 7 and EphA4 (A) Dose response curve for Compound 7. The binding of Compound 7 to EphA4 LBD was monitored by measuring the changes in anisotropy as it displaced the known binding peptide KYL from the active site. Kᵢ values are 0.50-0.66 μM in repeated experiments as measured by FPA. (B) EphA4-LBD was dissolved in 50 mM potassium phosphate buffer (pH 6.5), containing 100 mM NaCl. ITC were measured. Kd˜0.45 μM from this experiment as measured by ITC.

The Kᵢ and K_{d} measured by the ITC and FPA methods as described herein are shown in the following Table.

TABLE 2

| Comp. | Kᵢ by FPA | K_{d} by ITC |
|---|---|---|
| 1 | C | NT |
| 2 | NT | NT |
| 3 | A | NT |
| 4 | B | NT |
| 5 | B | B |
| 6 | B | NT |
| 7 | A | A |
| 8 | B | B |
| 9 | B | NT |
| 10 | B | A |
| 11 | C | NT |

A < 1 μM
1 μM ≤ B < 5 μM
5 μM ≤ C
NT: not tested

### Example II. Detection of Binding Between Compound 7 and EphA4-LBD by NMR Spectroscopy

NMR spectra of ¹⁵N-labeled EphA4-LBD were acquired on 700 MHz Bruker Avance spectrometer equipped with TCI cryoprobe. All NMR data were processed and analyzed using TOPSPIN2.1 (Bruker Biospin Corp., Billerica, Mass., USA) and SPARKY3.1 (University of California, San Francisco, Calif., USA). 2D-[¹⁵N, ¹H]-HSQC experiments were acquired using 32 scans with 2048 and 128 complex data points in the ¹H and ¹⁵N dimensions at 300 K. The binding is in slow exchange in the NMR time scale confirming the observed tight binding (Kd in the nM range) using the FPA and the ITC methods.

### Example III. Selectivity

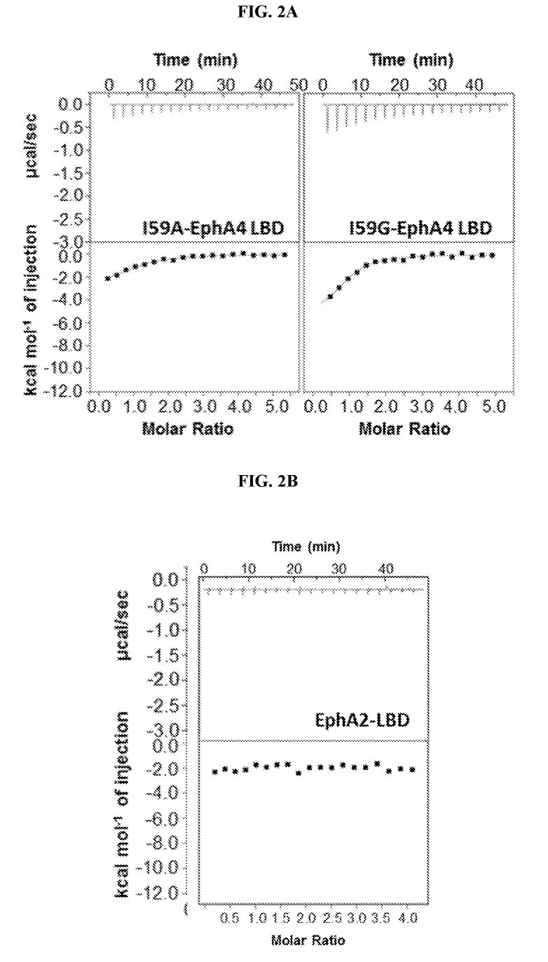

The EphA3 receptor is the closest in sequence to the EphA4, with only one critical mutation in the binding site: namely ephA4 residue Ile59 is a Glycine in EphA3. Hence, EphA4-LBD mutants 159A and 159G where prepared and the ability of Compound 7 to bind to these mutants was tested by ITC as described above. A marked loss in binding affinity of Compound 7 for these mutants indicating a selective binding for the EphA4 was observed. Similarly, using the same experimental conditions, binding to the EphA2 receptor was not observed (FIG. 2B).

FIG. 2. (A) ITC data for the interactions between Compound 7 and the indicated constructs. The Kd values are 9.09 μM for 159A mutant (left panel), 3.66 for the 159G mutant (right panel) and (B) ITC data for the interactions between Compound 7 and the EphA2 receptor.

### Example IV. In Vivo Efficacy in an Animal Model of ALS

To determine an approximate dose to be injected to SOD1(G93A)-mutant mice, 2 mice were initially treated postnatal day 60 until the endpoints with 30 mg/Kg of Compound 7 daily for several weeks intra-peritoneally and mice were observed for any signs of toxicity compared to untreated mice (n=2) receiving only vehicle as control. At the end of the pilot study, no adverse sign of toxicity were observed in the treated mice compared to the control mice. In addition, increased survival was observed with the treated mice suggesting that a more robust study could be conducted with a more sizable number of animals.

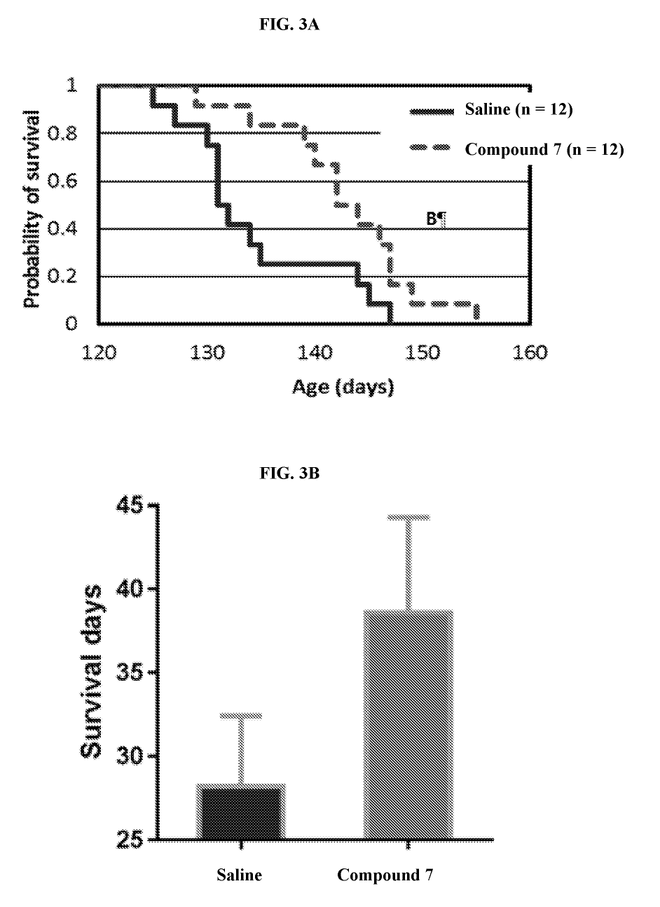

Equal number of SOD1(G93A)-mutant mice of the same gender from same litter were randomly divided into two groups (n=12). Mice were treated from postnatal day 60 until the endpoints by i.p. injections with either Compound 7 or the saline control. Daily treatment with 30 mg/kg of Compound 7 improved average life span from 134.3±7.2 days of control mice to 142.8±6.9 days of Compound 7-treated mice (p<0.01, Student's t-test). Compound 7 treatment altered disease duration. The average survival time from disease onset to end point were 28.2±4.2 and 38.6±5.7 days (p<0.01, Student's t-test) for control and Compound 7-treated mice, respectively. Kaplan-Meier survival plots revealed an increased survival of Compound 7-treated mice compared to control mice (FIG. 3).

FIG. 3. In vivo efficacy studies with Compound 7. (A) Cumulative probabilities of survival time of SOD1(G93A) mice treated with Compound 7 (in saline, 30 mg/Kg, i.p. daily) or saline alone. Compound 7 treatment prolonged survival without affecting the disease onset. (B) Treatment with compound Compound 7 increased the average survival time from disease onset to end point and prolonged overall life span in SOD1(G93A) mice, similar to what observed in the ephA4+/−mice. The data were analyzed using the Kaplan-Meier method. Animal studies. Equal number of SOD1(G93A)-mutant mice of the same gender from same litter were randomly divided into two groups. They were treated from postnatal day 60 until the endpoints by i.p. injections with either Compound 7 or the saline control. Compound 7 was dissolved in normal saline at a concentration of 2 mg/mL and sterilized by a 0.2 m filter. Mice were treated with Compound 7 every day, at a dose of 30 mg/kg of body weight. Animals were assessed daily for the time of onset of hind limb tremor and loss of splay reflex. The endpoint was designated as the point when the mouse could no longer roll over within 10 s after being pushed onto its side. At this stage, mice were killed. Disease onset and survival time were compared using the Kaplan-Meier method. Difference of survival time from disease onset to end point between Compound 7 and saline-treated mice was analyzed with Student's t-test. Values are given as mean±SD. Preliminary BBB penetration and PK studies were performed by Agilux (Cambridge, Mass.) and in house.

### Example V: Clinical Trial to Assess the Efficacy, Tolerability and Safety of Oral Administration of Compound 7 Compared to a Placebo in Subjects with ALS

The purpose of this study is to assess the efficacy, tolerability and safety of oral administration of Compound 7 compared to a placebo in subjects with ALS.

### Study Type: Interventional

### Study Design: Allocation: Randomized

### Endpoint Classification: Safety/Efficacy Study

### Intervention Model: Parallel Assignment

### Masking: Double Blind (Subject, Caregiver, Investigator, Outcomes Assessor)

### Primary Purpose: Treatment

Primary Outcome Measures: Change in ALS Functional Rating Score (ALSFRS-R slope) [Time Frame: 52 weeks] [Designated as safety issue: No]
Secondary Outcome Measures: Time from baseline to the first occurrence of either death, tracheostomy or permanent assisted ventilation. [Time Frame: 52 weeks] [Designated as safety issue: No]

| Arms | Assigned Interventions |
|---|---|
| Experimental: Compound 7 | Drug: Compound 7 |
| 3 times a day | 3 times per day, 52 weeks |
| Placebo Comparator: Placebo | Other: placebo |
| placebo 3 times per day | placebo, 3 times a day, for 52 weeks |

### Eligibility:

### Ages Eligible for Study: 18 Years to 80 Years

### Genders Eligible for Study: Both

### Accepts Healthy Volunteers: No

### Criteria:

Subjects with sporadic or familial ALS classified as definite, probable, or laboratory-supported probable ALS according to the revised El Escorial criteria.

### Inclusion Criteria:

1. Diagnosis of definite or probable ALS in accordance with the El-Escorial criteria.
2. Subject has experienced his/her first ALS symptoms within 3 years prior to the screening visit.
3. Slow VC test equal to or greater than 70% of the predicted value.
4. The sum of the 3 respiratory items on the ALSFRS-R must total at least 10 points.
5. Subjects taking riluzole must be on a stable dose for at least 8 weeks prior to screening visit.
6. Ages 18-80 (inclusive)

### Exclusion Criteria:

1. The use of invasive or non-invasive ventilation.
2. Subject having undergone gastrostomy.
3. Subject with any clinically significant or unstable medical condition.
4. Subject participating in any other investigational drug trial or using investigational drug (within 12 weeks prior to screening and thereafter).
5. Females who are pregnant or nursing.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

We claim:

1. A compound of formula (I), or a pharmaceutically acceptable salt, solvate, or stereoisomer thereof: 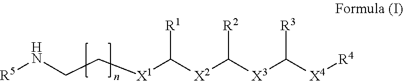
wherein
X¹, X², X³, and X⁴ are each independently —C(═O)NH—, —NHC(═O)—, —S(═O)₂NH—, —O—, —CH₂—, —CH₂CH₂—, —OCH₂—, —CH₂O—, —S—, —S(═O)₂—, or —C(═O)NHS(═O)₂—;
R¹, R², R³, and R⁴ are each independently alkyl, heterocycloalkyl, or cycloalkyl; wherein the alkyl, heterocycloalkyl, and cycloalkyl are optionally substituted with one of more R^{a};
R⁵ is —H, —CH₃, —C₂H₅, —C(═O)H, —C(═O)aryl, —C(═O)alkyl, or —C(═O)(CH₂CH₂O)ₖCH₃;
R⁶ is —H, halogen, alkyl, haloalkyl, alkoxy, haloalkoxy, —OH, —NH₂, S(═O)₂NH₂, —C(═O)NH₂, —NHC(═O)H, —NHC(═O)CH₃, —NO₂, —S(═O)CH₃, —NHS(═O)₂CH₃, —OCH₂OCH₃, —OCH₂CH₂OCH₃, —NHC(═O)H, or —NHC(═O)CH₃;
R^{a} is R⁶, aryl or heteroaryl; wherein the aryl or heteroaryl are optionally substituted with one or more R^{b};
R^{b} is —H, halogen, alkyl, haloalkyl, alkoxy, haloalkoxy, —OH, —NH₂, —S(═O)₂NH₂, —C(═O)NH₂, —NHC(═O)H, —NHC(═O)CH₃, —NO₂, —S(═O)CH₃, —NHS(═O)₂CH₃, —OCH₂OCH₃, or —OCH₂CH₂OCH₃;
n is 0-4; and
k is 1-1000.

2. The compound of claim 1, or a pharmaceutically acceptable salt, solvate, or stereoisomer thereof, wherein n is 2-4.

3. The compound of claim 1 or 2, or a pharmaceutically acceptable salt, solvate, or stereoisomer thereof, wherein R¹, R², R³, and R⁴ are each independently alkyl substituted with one of more R^{a}.

4. The compound of any one of claims 1-3, or a pharmaceutically acceptable salt, solvate, or stereoisomer thereof, wherein R¹, R², R³, and R⁴ are each independently —CH₃ or —CH₂CH₃ substituted with one of more R^{a}.

5. The compound of any one of claims 1-4, or a pharmaceutically acceptable salt, solvate, or stereoisomer thereof, wherein R¹, R², R³, and R⁴ are each independently —CH₃ or —CH₂CH₃ substituted with one R^{a}.

6. The compound of any one of claims 1-4, or a pharmaceutically acceptable salt, solvate, or stereoisomer thereof, wherein R¹, R², R³, and R⁴ are each independently —CH₃ or —CH₂CH₃ substituted with two R^{a}.

7. The compound of any one of claims 1-4, or a pharmaceutically acceptable salt, solvate, or stereoisomer thereof, wherein R¹, R², and R³ are each independently —CH₃ substituted with one R^{a}.

8. The compound of any one of claims 1-4, or a pharmaceutically acceptable salt, solvate, or stereoisomer thereof, wherein R⁴ is —CH₂CH₃ substituted with two R^{a}.

9. The compound of any one of claims 1-7, or a pharmaceutically acceptable salt, solvate, or stereoisomer thereof, wherein R¹ is alkyl substituted with a heteroaryl optionally substituted with one of more R^{b}.

10. The compound of any one of claims 1-7, or a pharmaceutically acceptable salt, solvate, or stereoisomer thereof, wherein R¹ is alkyl substituted with an indolyl optionally substituted with one of more R^{b}.

11. The compound of any one of claims 1-7, or a pharmaceutically acceptable salt, solvate, or stereoisomer thereof, wherein R¹ is alkyl substituted with an indolyl optionally substituted with one R^{b}.

12. The compound of any one of claims 1-7, or a pharmaceutically acceptable salt, solvate, or stereoisomer thereof, wherein R² is alkyl substituted with an aryl optionally substituted with one of more R^{b}.

13. The compound of any one of claims 1-7, or a pharmaceutically acceptable salt, solvate, or stereoisomer thereof, wherein R² is alkyl substituted with a phenyl optionally substituted with one R^{b}.

14. The compound of any one of claims 1-7, or a pharmaceutically acceptable salt, solvate, or stereoisomer thereof, wherein R² is alkyl substituted with a phenyl optionally substituted with one of more R^{b}.

15. The compound of any one of claims 1-7, or a pharmaceutically acceptable salt, solvate, or stereoisomer thereof, wherein R³ is alkyl substituted with a heteroaryl optionally substituted with one of more R^{b}.

16. The compound of any one of claims 1-7, or a pharmaceutically acceptable salt, solvate, or stereoisomer thereof, wherein R³ is alkyl substituted with a pyridyl optionally substituted with one of more R^{b}.

17. The compound of any one of claims 1-7, or a pharmaceutically acceptable salt, solvate, or stereoisomer thereof, wherein R³ is alkyl substituted with a pyridyl optionally substituted with one R^{b}.

18. The compound of any one of claims 1-6 or 8, or a pharmaceutically acceptable salt, solvate, or stereoisomer thereof, wherein R⁴ is alkyl substituted with a heteroaryl optionally substituted with one of more R^{b}.

19. The compound of any one of claims 1-6 or 8, or a pharmaceutically acceptable salt, solvate, or stereoisomer thereof, wherein R⁴ is alkyl substituted with an indolyl optionally substituted with one of more R^{b}.

20. The compound of any one of claims 1-6 or 8, or a pharmaceutically acceptable salt, solvate, or stereoisomer thereof, wherein R⁴ is alkyl substituted with R⁶ and with a heteroaryl optionally substituted with one of more R^{b}.

21. The compound of any one of claims 1-6 or 8, or a pharmaceutically acceptable salt, solvate, or stereoisomer thereof, wherein R⁴ is alkyl substituted with R⁶ and with an indolyl optionally substituted with one of more R^{b}.

22. The compound of any one of claims 1-6 or 8, or a pharmaceutically acceptable salt, solvate, or stereoisomer thereof, wherein R⁴ is alkyl substituted with R⁶ and with an indolyl optionally substituted with one R^{b}.

23. The compound of any one of claims 1-22, or a pharmaceutically acceptable salt, solvate, or stereoisomer thereof, wherein R^{b} is —H, —OH, —OCH₃, —OC₂H₅, —F, —Cl, —Br, —I, —NH₂, —S(═O)₂NH₂, —CF₃, —C(═O)NH₂, —NHC(═O)H, —NHC(═O)CH₃, —NO₂, —S(═O)CH₃, —NHS(═O)₂CH₃, —OCF₃, —OCH(CH₃)₂, —CH(CH₃)₂, —CH₂CH₂CH₃, —OCH₂CH₂CH₃, —OCH₂OCH₃, or —OCH₂CH₂OCH₃.

24. The compound of any one of claims 1-23, or a pharmaceutically acceptable salt, solvate, or stereoisomer thereof, wherein R^{b} is —H, —OH, —OCH₃, —F, or —Cl.

25. The compound of any one of claims 1-24, or a pharmaceutically acceptable salt, solvate, or stereoisomer thereof, wherein R⁶ is —H, —OH, —OCH₃, —OC₂H₅, —F, —Cl, —Br, —I, —NH₂, S(═O)₂NH₂, —CF₃, —C(═O)NH₂, —NHC(═O)H, —NHC(═O)CH₃, —NO₂, —S(═O)CH₃, —NHS(═O)₂CH₃, —OCF₃, —OCH(CH₃)₂, —CH(CH₃)₂, —CH₂CH₂CH₃, —OCH₂CH₂CH₃, —OCH₂OCH₃, —OCH₂CH₂OCH₃, —NHC(═O)H, or —NHC(═O)CH₃.

26. The compound of any one of claims 1-25, or a pharmaceutically acceptable salt, solvate, or stereoisomer thereof, wherein R⁶ is —H or —C(═O)NH₂.

27. The compound of any one of claims 1-26, or a pharmaceutically acceptable salt, solvate, or stereoisomer thereof, wherein X¹, X², X³, and X⁴ are each independently —C(═O)NH— or —NHC(═O)—.

28. The compound of any one of claims 1-27, or a pharmaceutically acceptable salt, solvate, or stereoisomer thereof, wherein X¹, X², X³, and X⁴ are each —C(═O)NH—.

29. The compounds of any one of claims 1-28, or a pharmaceutically acceptable salt, solvate, or stereoisomer thereof, wherein the compound of Formula (I) has the Formula (Ia): 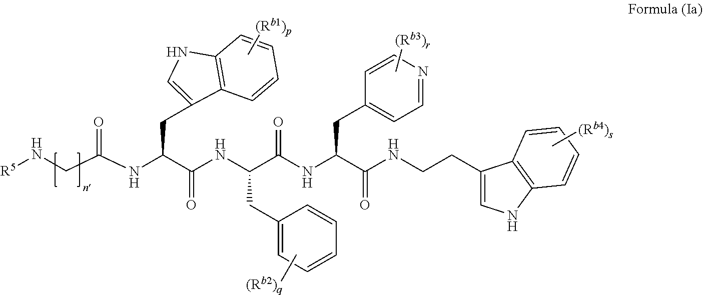
wherein
R⁵ is —H, —CH₃, —C₂H₅, —C(═O)H, —C(═O)aryl, —C(═O)alkyl, or —C(═O)(CH₂CH₂O)ₖCH₃;
R^{b1}, R^{b2}, R^{b3}, and R^{b4} are independently —H, —OH, —OCH₃, —OC₂H₅, —F, —Cl, —Br, —I, —NH₂, —S(═O)₂NH₂, —CF₃, —C(═O)NH₂, —NHC(═O)H, —NHC(═O)CH₃, —NO₂, —S(═O)CH₃, —NHS(═O)₂CH₃, —OCF₃, —OCH(CH₃)₂, —CH(CH₃)₂, —CH₂CH₂CH₃, —OCH₂CH₂CH₃, —OCH₂OCH₃, or —OCH₂CH₂OCH₃;
n′ is 0-4;
k is 1-1000; and
p, q, r, and s are each independently 1-3.

30. The compounds any one of claims 1-28, or a pharmaceutically acceptable salt, solvate, or stereoisomer thereof, wherein the compound of Formula (I) has the Formula (Ib): 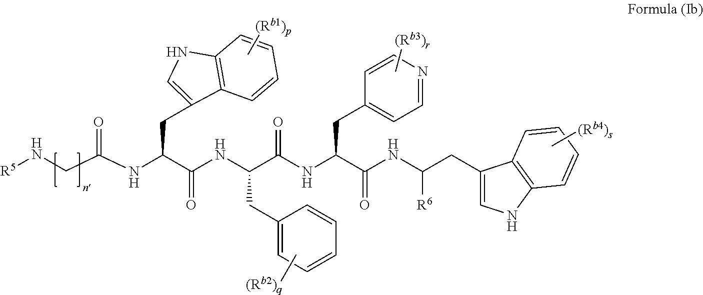
wherein
R⁵ is —H, —CH₃, —C₂H₅, —C(═O)H, —C(═O)aryl, —C(═O)alkyl, or —C(═O)(CH₂CH₂O)ₖCH₃;
R⁶ is —H, —OH, —OCH₃, —OC₂H₅, —F, —Cl, —Br, —I, —NH₂, S(═O)₂NH₂, —CF₃, —C(═O)NH₂, —NHC(═O)H, —NHC(═O)CH₃, —NO₂, —S(═O)CH₃, —NHS(═O)₂CH₃, —OCF₃, —OCH(CH₃)₂, —CH(CH₃)₂, —CH₂CH₂CH₃, —OCH₂CH₂CH₃, —OCH₂OCH₃, —OCH₂CH₂OCH₃, —NHC(═O)H, or —NHC(═O)CH₃;
R^{b1}, R^{b2}, R^{b3}, and R^{b4} are independently —H, —OH, —OCH₃, —OC₂H₅, —F, —Cl, —Br, —I, —NH₂, —S(═O)₂NH₂, —CF₃, —C(═O)NH₂, —NHC(═O)H, —NHC(═O)CH₃, —NO₂, —S(═O)CH₃, —NHS(═O)₂CH₃, —OCF₃, —OCH(CH₃)₂, —CH(CH₃)₂, —CH₂CH₂CH₃, —OCH₂CH₂CH₃, —OCH₂OCH₃, or —OCH₂CH₂OCH₃;
n′ is 0-4;
k is 1-1000; and
p, q, r, and s are each independently 1-3.

31. The compound of claim 30, or a pharmaceutically acceptable salt, solvate, or stereoisomer thereof, wherein R⁶ is —H or —C(═O)NH₂.

32. The compound of any one of claims 29-31, or a pharmaceutically acceptable salt, solvate, or stereoisomer thereof, wherein n′ is 1.

33. The compounds any one of claims 29-31, or a pharmaceutically acceptable salt, solvate, or stereoisomer thereof, wherein n′ is 3.

34. The compounds any one of claims 29-31, or a pharmaceutically acceptable salt, solvate, or stereoisomer thereof, wherein n′ is 4.

35. The compound of any one of claims 29-34, or a pharmaceutically acceptable salt, solvate, or stereoisomer thereof, wherein R^{b1}, R^{b}2, R^{b3}, and R^{b4} are independently —H, —OH, —OCH₃, —F, or —Cl.

36. The compound of any one of claims 29-35, or a pharmaceutically acceptable salt, solvate, or stereoisomer thereof, wherein p is 1; and R^{b1} is —OH.

37. The compound of any one of claims 29-35, or a pharmaceutically acceptable salt, solvate, or stereoisomer thereof, wherein q is 1; and R^{b2} —Cl.

38. The compound of any one of claims 29-35, or a pharmaceutically acceptable salt, solvate, or stereoisomer thereof, wherein r is 1; and R^{b3} is —H.

39. The compound of any one of claims 29-35, or a pharmaceutically acceptable salt, solvate, or stereoisomer thereof, wherein s is 1; and R^{b4} is —H, —OH, —OCH₃, —F, or —Cl.

40. The compound of any one of claims 1-39, or a pharmaceutically acceptable salt, solvate, or stereoisomer thereof, wherein R⁵ is —H.

41. The compound of any one of claims 1-39, or a pharmaceutically acceptable salt, solvate, or stereoisomer thereof, wherein R⁵ is —C(═O)(CH₂CH₂O)ₖCH₃.

42. The compound of claim 41, or a pharmaceutically acceptable salt, solvate, or stereoisomer thereof, wherein k is 1 to 100.

43. The compound of claim 41, or a pharmaceutically acceptable salt, solvate, or stereoisomer thereof, wherein k is 100 to 500.

44. The compound of claim 41, or a pharmaceutically acceptable salt, solvate, or stereoisomer thereof, wherein k is 500 to 1000.

45. The compound of claim 1, or a pharmaceutically acceptable salt, solvate, or stereoisomer thereof, represented by the following formula:

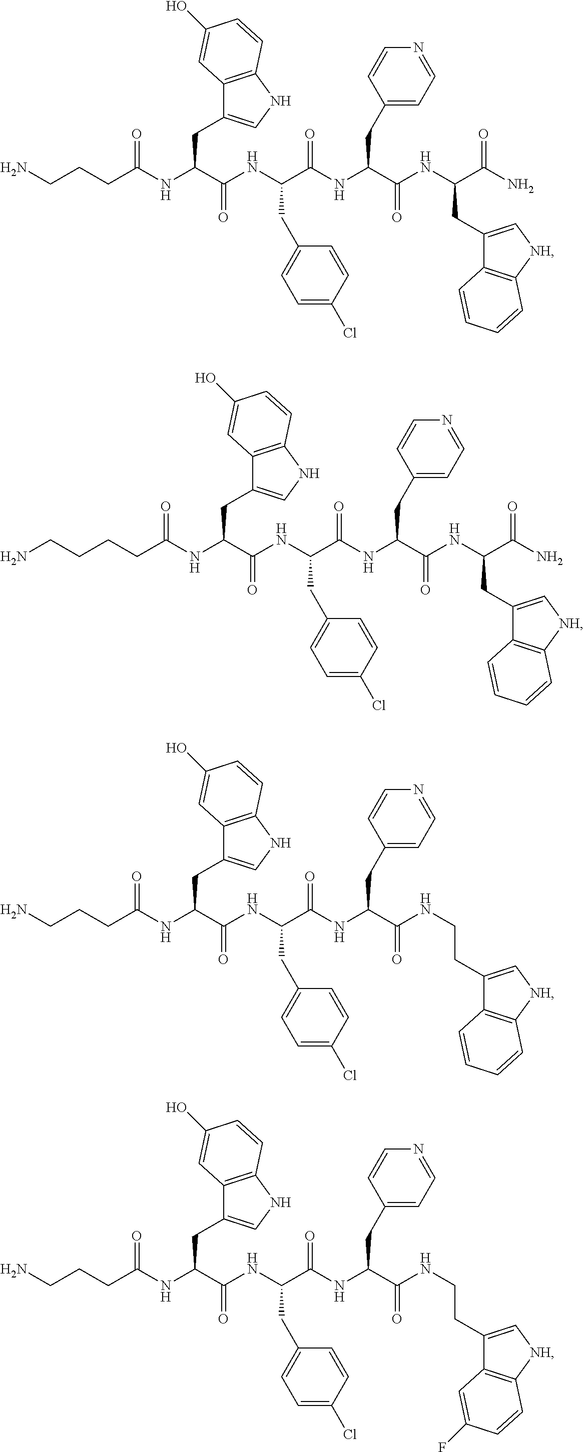 [SMILES: NCCCC(=O)N[C@@H](Cc1c[nH]c2ccc(O)cc12)C(=O)N[C@@H](Cc1ccc(Cl)cc1)C(=O)N[C@@H](Cc1ccncc1)C(=O)N[C@H](Cc1c[nH]c2ccccc12)C(N)=O ; NCCCCC(=O)N[C@@H](Cc1c[nH]c2ccc(O)cc12)C(=O)N[C@@H](Cc1ccc(Cl)cc1)C(=O)N[C@@H](Cc1ccncc1)C(=O)N[C@H](Cc1c[nH]c2ccccc12)C(N)=O ; NCCCC(=O)N[C@@H](Cc1c[nH]c2ccc(O)cc12)C(=O)N[C@@H](Cc1ccc(Cl)cc1)C(=O)N[C@@H](Cc1ccncc1)C(=O)NCCc1c[nH]c2ccccc12 ; NCCCC(=O)N[C@@H](Cc1c[nH]c2ccc(O)cc12)C(=O)N[C@@H](Cc1ccc(Cl)cc1)C(=O)N[C@@H](Cc1ccncc1)C(=O)NCCc1c[nH]c2ccc(F)cc12]

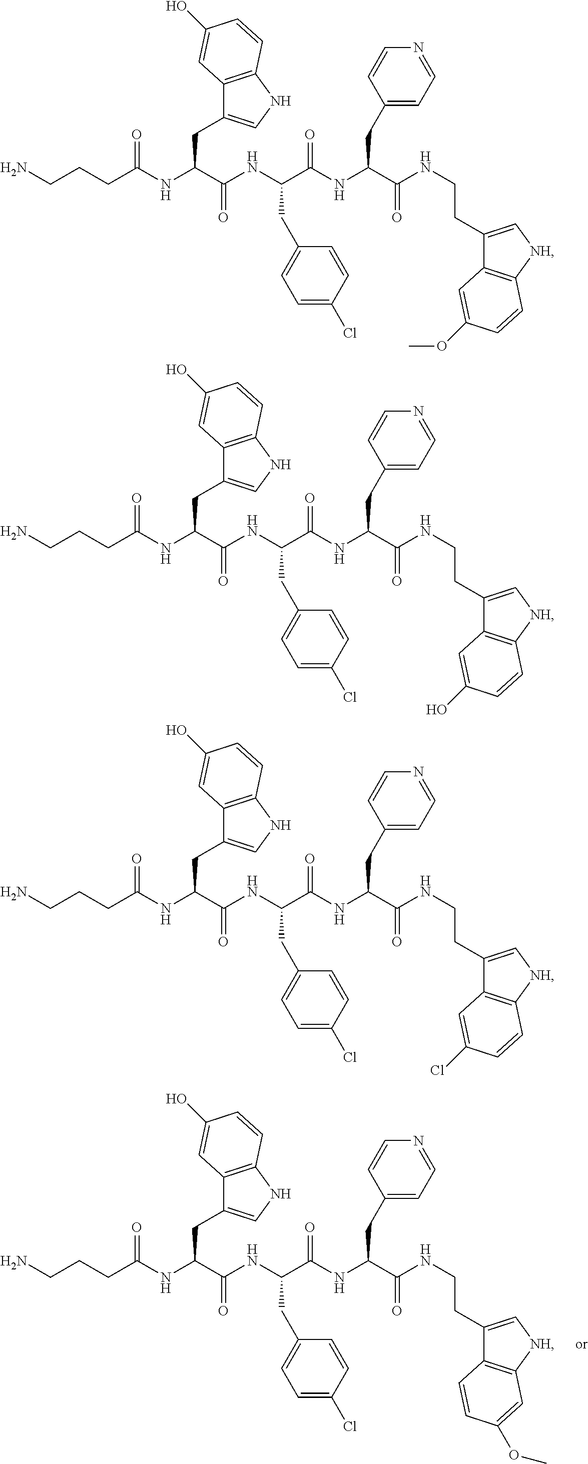 [SMILES: COc1ccc2[nH]cc(CCNC(=O)[C@H](Cc3ccncc3)NC(=O)[C@H](Cc3ccc(Cl)cc3)NC(=O)[C@H](Cc3c[nH]c4ccc(O)cc34)NC(=O)CCCN)c2c1 ; NCCCC(=O)N[C@@H](Cc1c[nH]c2ccc(O)cc12)C(=O)N[C@@H](Cc1ccc(Cl)cc1)C(=O)N[C@@H](Cc1ccncc1)C(=O)NCCc1c[nH]c2ccc(O)cc12 ; NCCCC(=O)N[C@@H](Cc1c[nH]c2ccc(O)cc12)C(=O)N[C@@H](Cc1ccc(Cl)cc1)C(=O)N[C@@H](Cc1ccncc1)C(=O)NCCc1c[nH]c2ccc(Cl)cc12 ; COc1ccc2c(CCNC(=O)[C@H](Cc3ccncc3)NC(=O)[C@H](Cc3ccc(Cl)cc3)NC(=O)[C@H](Cc3c[nH]c4ccc(O)cc34)NC(=O)CCCN)c[nH]c2c1]

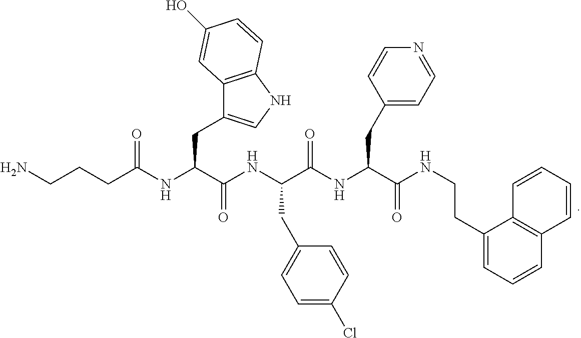 [SMILES: NCCCC(=O)N[C@@H](Cc1c[nH]c2ccc(O)cc12)C(=O)N[C@@H](Cc1ccc(Cl)cc1)C(=O)N[C@@H](Cc1ccncc1)C(=O)NCCc1cccc2ccccc12]

46. A pharmaceutical composition comprising a compound of any one of claims 1-45 and a pharmaceutically acceptable excipient.

47. A pharmaceutical composition comprising a compound of any one of claims 1-45 and a pharmaceutically acceptable excipient, wherein the pharmaceutical composition is in the form of nanoparticles.

48. A method of treating amyotrophic lateral sclerosis (ALS) in a subject in need thereof comprising administering to the subject a therapeutically effective amount of a compound of any one of claims 1-45.

49. A method of treating amyotrophic lateral sclerosis (ALS) in a subject in need thereof comprising administering to the subject a pharmaceutical composition of claim 46 or 47.

50. A method of treating abnormal blood clotting in a subject in need thereof comprising administering to the subject a therapeutically effective amount of a compound of any one of claims 1-45.

51. A method of treating abnormal blood clotting in a subject in need thereof comprising administering to the subject a pharmaceutical composition of claim 46 or 47.

52. A method of treating gastric cancer in a subject in need thereof comprising administering to the subject a therapeutically effective amount of a compound of any one of claims 1-45.

53. A method of treating gastric cancer in a subject in need thereof comprising administering to the subject a pharmaceutical composition of claim 46 or 47.

54. A method of treating spinal cord injury in a subject in need thereof comprising administering to the subject a therapeutically effective amount of a compound of any one of claims 1-45.

55. A method of treating spinal cord injury in a subject in need thereof comprising administering to the subject a pharmaceutical composition of claim 46 or 47.

56. A method of treating Alzheimer's disease (AD) in a subject in need thereof comprising administering to the subject a therapeutically effective amount of a compound of any one of claims 1-45.

57. A method of treating Alzheimer's disease (AD) in a subject in need thereof comprising administering to the subject a pharmaceutical composition of claim 46 or 47.

58. A method of treating traumatic brain injury in a subject in need thereof comprising administering to the subject a therapeutically effective amount of a compound of any one of claims 1-45.

59. A method of treating traumatic brain injury in a subject in need thereof comprising administering to the subject a pharmaceutical composition of claim 46 or 47.

60. The method of any one of claims 48-59, wherein the compound of any one of claims 1-45 or the pharmaceutical composition of claim 46 or 47 is administered intranasally, orally, parenterally, intravenously, intraperitoneally, intramuscularly, topically or subcutaneously.
